(19) United States
(12) Patent Application Publication
(10) Pub. No.: US 2014/0058741 A1
(43) Pub. Date: Feb. 27, 2014

(54) AUTOMATED SYSTEM AND METHOD FOR MEDICAL CARE SELECTION

(71) Applicant: National Consolidated Technologies, LLC, Bloomfield Hills, MI (US)

(72) Inventor: Donald G. Bronn, Bloomfield Hills, MI (US)

(73) Assignee: National Consolidated Technologies, LLC, Bloomfield Hills, MI (US)

(21) Appl. No.: 13/852,271

(22) Filed: Mar. 28, 2013

Related U.S. Application Data
(63) Continuation of application No. 12/016,963, filed on Jan. 18, 2008, now abandoned.
(60) Provisional application No. 60/886,088, filed on Jan. 22, 2007.

(51) Int. Cl. G06F 19/00

(52) U.S. Cl. CPC G06F 19/3418; G06F 19/345
USPC 705/2

(57) ABSTRACT

Provided herein is a computerized method of managing medical care through communication between a Digital Board of Medical Experts (DBME), a physician, a health insurance carrier, one or more medical facilities, and a patient and providing a diagnostic and/or therapeutic recommendation, the method comprising the following steps: i) providing a DBME core comprised of modules and algorithms for processing medical data and providing diagnostic and/or therapeutic recommendations; ii) providing a Physician Action Module (PAM) whereby a physician provides a clinical index of suspicion (CIS) through a clinical index of suspicion selection algorithm (CISSA) of the PAM to the DBME—for obtaining a recommendation for hi tech diagnostic studies and/or treatment; and iii) processing through the DBME information from the CISSA and providing a diagnostic recommendation from the diagnostic recommendation algorithm (DRA) of the Diagnostic Module (DM) of the DBME for hi tech diagnostic studies and/or a therapeutic recommendation from the therapeutic recommendation algorithm (TRA) of the Therapeutic Module (TM) of the DBME for treatment.

### CROSS-REFERENCE

This application claims the benefit of U.S. Provisional Application No. 60/886,088, filed Jan. 22, 2007, and U.S. Utility application Ser. No. 12/016,963, filed Jan. 18, 2008, the contents of which are incorporated herein by reference in their entirety.

### BACKGROUND OF THE INVENTION

The United States spends more on healthcare than any other nation in the world both on a per capita basis and as a portion of gross domestic product. Furthermore, healthcare costs in the United States continue to increase at a staggering pace of 10-12% per year. In 2000, the U.S. spent $1.3 trillion on healthcare. In 2006, the annual cost of healthcare had risen to $2 trillion. It is expected to reach an annual cost of $2.6 trillion in 2010 and will exceed $4 trillion in 2016. Currently our healthcare spending is 15% of GDP and is expected to reach 19.6% of GDP by 2016.

Most Americans (84%) pay for healthcare costs through health insurance obtained either through their employer (60%), purchased individually (9%), through government programs (40%) or a combination thereof, accounting for the overlap in percentages. The U.S. Government is the largest insurer of healthcare in the United States. However, there are approximately 47 million Americans that are uninsured (16% of the population). Uninsured individuals are at personal risk for expensive medical costs and medical bills are the overwhelming reason for personal bankruptcies in the United States.

Currently, advocates for healthcare reform have preferred universal private health insurance coverage as opposed to a government run program of socialized medicine like the Canadian system that depends heavily on rationing high-end healthcare services. Mandated universal private health insurance coverage for all United States citizens would be supported by tax credits and other government supplementation for the currently uninsured. Nevertheless, current abuses of the healthcare system (overuse, underuse and misuse) that contribute to rising healthcare costs would not be addressed by assured universal private health insurance coverage.

The United States healthcare system has been criticized not only for its expense but also for issues of access, efficiency and wide variations in quality. In 2000, the World Health Organization has ranked the U.S. Healthcare System as 37^{th} for overall performance with the overall health of Americans as 72^{nd} among 191 member nations. The study did not take into account mitigating factors such as a generalized trend to obesity and other excesses of an abundant society whose life-style is promotional of habits that are incompatible with the prevention of diseases such as diabetes, heart disease and cancer. For example, Americans consume an average of 120 pounds of sugar per capita and as a result lead the world in the incidence of diabetes.

On the other hand, the American healthcare system leads the world in individual survival time after a diagnosis of cancer or symptomatic heart disease. It also leads the world in medical research, medical publications, development of pharmaceuticals and medical devices for both diagnosis and therapy, and other diagnostic and therapeutic innovations. Government leaders, monarchs, billionaires and other celebrated notables from all over the world routinely travel to the United States for the treatment of life-threatening diseases. The general consensus is that the United States has the best medical diagnostics and therapeutics for advanced diseases in the world if you have the resources, influence and knowledge to obtain them.

Therefore, any meaningful improvement in the U.S. healthcare system must not inhibit the incentives that promote and reward medical innovation in a privatized healthcare system but rather improve the mechanisms of the U.S. healthcare delivery system in terms of equal access to the best diagnostics and therapeutics, improvement in diagnostic and therapeutic efficiencies, and improvements in the uniformity of quality of care across the entire healthcare system.

At first glance, it would seem that such improvements would contribute to even further increases in healthcare costs. In fact, the opposite is true. Under the current delivery system of healthcare, there are massive inefficiencies with huge inconsistencies of quality contributing to poor outcomes from ill-timed or unnecessary procedures resulting in increased complications and treatment failures. It is estimated that uniform improvements in the management of just two disease entities such as cancer and heart disease would result in a cost savings in excess of $80 billion annually.

It has been suggested that conversion of paper based medical records to electronic medical records (EMR) would help identify which medical practices are more effective and less costly. This would help standardize healthcare delivery to a higher level of quality (“best practices”) and thus improve the current wide variations in quality, efficiency and access. It has been estimated that over 90% of patients records in physician offices are paper based.

Physician offices are essentially a cottage industry and thus have been reluctant to bear the high cost of EMR conversion due to a myriad of logistical factors including cumbersome high volume data management, rapid changes in EMR technology with impending obsolescence of existing systems and compatibility issues with other EMR systems at hospitals, medical facilities and health insurance carriers. Thus, none of the stakeholders within a private healthcare system have been willing to step up and absorb the massive costs necessary for comprehensive EMR conversions within physician offices and clinics.

There remains a need for methods and systems than efficiently process individual patient information while providing state of the art assessment and diagnostic and therapeutic recommendations.

Any successful modification to the U.S. healthcare delivery system would have to positively impact the needs and interests of four principal stakeholders: the patient, the physician, the medical facility and the health insurance carrier. Thus, it is desirable to provide tools that efficiently link physicians, experts, treatment and diagnostic facilities, insurance carriers and patients.

### SUMMARY OF THE INVENTION

The present invention provides a computerized method of managing medical care through communication between a Digital Board of Medical Experts (DBME), a physician, a health insurance carrier, one or more medical facilities, and a patient and providing a diagnostic and/or therapeutic recommendation, the method comprising the following steps: i) providing a DBME core comprised of modules and algorithms for processing medical data and providing diagnostic and/or therapeutic recommendations; ii) providing a Physician Action Module (PAM) whereby a physician provides a clinical index of suspicion (CIS) through a clinical index of suspicion selection algorithm (CISSA) of the PAM to the DBME—for obtaining a recommendation for hi tech diagnostic studies and/or treatment; and iii) processing through the DBME information from the CISSA and providing a diagnostic recommendation from the diagnostic recommendation algorithm (DRA) of the Diagnostic Module (DM) of the DBME for hi tech diagnostic studies and/or a therapeutic recommendation from the therapeutic recommendation algorithm (TRA) of the Therapeutic Module (TM) of the DBME for treatment.

In one embodiment, the DBME internal core comprises one or more of a Diagnostic Module (DM), a Therapeutic Module (TM), a Digital Radiological Reading and Review Module (DRRM), a Continuous Medical Education Module (CMEM), a Monetization Module (MM), a Medical Malpractice Risk Management Module (MMRMM), a Privacy Compliance Module (PCM), an Algorithm Boards Sub-specialist Selection Module (ABSSM) and the DBME external core comprises one or more of a Patient Information Module (PIM), a Physician Action Module (PAM), a Health Insurance Authorization Module (IAM) and a Medical Facility Action Module (MFAM).

In another embodiment, the PAM comprises a clinical index of suspicion selection algorithm (CISSA), a deviation factors submission algorithm (DFSA), a therapeutic specialist qualification and assignment algorithm (TSQAA), and a physician targeted advertising algorithm (PTAA).

Another embodiment provides a method further comprising selection by the physician of a CIS from a CIS menu processed through the CISSA.

In another embodiment, the PIM comprises a layman's terms conversion algorithm (LTCA), a targeted advertising algorithm for patients (TAA), and a patient's credit processing and cost comparison for co-pay/deductible algorithm (PCPA).

Yet another embodiment provides a method wherein the DM comprises one or more of a diagnostic recommendation algorithm (DRA), diagnostic consensus review algorithm (DCRA), a diagnostic consensus improvement algorithm (DCIA), and a diagnostic time sensitive response algorithm (DTSRA).

In another embodiment, the TM comprises one or more of a therapeutic recommendation algorithm (TRA), a therapeutic consensus review algorithm (TCRA), a therapeutic consensus improvement algorithm (TCIA), a prevention recommendation algorithm (PRA), and a therapeutic time sensitive response algorithm (TTSRA).

In still another embodiment the MFAM comprises one or more of a facility and personnel qualification algorithm (FPQA), a specialist qualification algorithm (SQA), a scheduling algorithm (SA), and an equipment utilization algorithm (EUA).

In still another embodiment the DRRM comprises one or more of a test quality assessment algorithm (TQAA), a test reading quality assessment algorithm (TRQAA), a payment proration algorithm (PPA), a performance tracking algorithm (PTA), and a reading specialist qualification and assignment algorithm (RSQAA).

In still another embodiment, the IAM comprises a payment approval algorithm (PAA) and/or a billing algorithm (BA).

In still another embodiment the CIS comprises a presenting complaint.

In one embodiment the presenting complaint comprises a chief presenting complaint.

In another embodiment the presenting complaint comprises a secondary presenting complaint.

In a further embodiment the CIS comprises a physician adjusted chief complaint.

In still another embodiment the CIS comprises symptomatology derived factors.

In one embodiment the symptomatology derived factors are based on patient complaints.

In another embodiment the CIS comprises past medical history factors.

In one embodiment the past medical history comprises prior treatments.

In a further embodiment the prior treatment comprises one or more of non-surgical treatment and surgical treatment.

In one embodiment the non-surgical treatment comprises one or more of non-invasive procedure and medications.

In yet another embodiment the CIS comprises physical examination findings.

In still another embodiment the CIS comprises results of laboratory tests.

In a further embodiment the laboratory tests comprise testing body fluids.

In one embodiment the body fluid is selected from blood, urine, spinal fluid, sputum, or other types of body fluids.

In yet another embodiment the CIS comprises the results of imaging test.

In still another embodiment the CIS comprises the results of cellular or tissue pathology findings.

In a further embodiment the imaging tests comprise X-ray, radionuclear and ultrasound.

In still another embodiment the CIS comprises asymptomatic profile.

In still another embodiment the CIS comprises a genetic profile.

In a further embodiment the CIS comprises environmental profile.

In a further embodiment the CIS comprises a behavioral profile.

In one embodiment the behavioral profile comprises alcohol drinking habits, nicotine intake habits; narcotic or other addictive substance use.

In still another embodiment the CIS comprises family history profile.

In a further embodiment the CIS comprises a proposed hi tech diagnostic test.

In one embodiment the DRA comprises determining an overlay fidelity index (OFI) between the CIS provided by the physician and DBME defined factors.

In another embodiment the overlay fidelity index required by the DRA is determined based on the type of test, the invasiveness of the test, risk to the patient associated with the test and cost of the test.

In still another embodiment the overlay fidelity index required by the DRA is adjusted based on cost effectiveness data.

In still another embodiment the overlay fidelity index is adjusted based on predetermined percentage of false positive and/or false negative outcomes.

In still another embodiment the cost effectiveness data is based on CIS appropriateness, diagnostic outcomes, and cost data.

In one embodiment the DRA provides a recommendation based on a CIS having an overlay fidelity index of 50% or greater.

In another embodiment the DRA comprises requests for additional CIS factors based on a threshold overlay fidelity index.

In still another embodiment factors in the CIS comprise weighting factors as adjusted by the OFI.

In a further embodiment the high tech diagnostic test comprises a non-virtual invasive procedure.

In still another embodiment the invasive procedure is selected from a colonoscopy, cystoscopy, arteriography, cholecystography, endoscopy, laparoscopy, and mediastinoscopy or other methods requiring visualization of internal organs.

In a further embodiment the high tech diagnostic test comprises a non-invasive procedure.

In yet another embodiment a non-invasive procedure is selected from CAT scans, radionuclear scans, PET scans, MRI, and ultrasound imaging.

In still another embodiment, the DM and TM of the DBME are formed by recommendation algorithms (DRA, TRA, PRA) developed by experts from major Universities and Clinics, who are thought leaders and recognized in their sub-specialty fields of medicine who are identified and chosen through the ABSSM.

In one embodiment the experts are organized in DCRA, DRA, TRA, TCRA and RSQAA boards.

In a further embodiment the algorithms are digitally linked for immediate access for diagnostic and therapeutic evaluations and recommendations.

In still another embodiment the DBME comprises algorithms for clinical presentations that have high impact probability for better outcomes with lower costs.

In still another embodiment the DBME comprises algorithms supported by a board of experts grouped according to a set of subspecialties.

In a further embodiment the experts maintain the integrity and quality of the algorithm.

In still another embodiment the experts are selected through the ABSSM based on their publications, lectures, clinical experience, faculty affiliations, positions within medical specialty colleges and associations, government agencies, national and international bodies, foundations, clinics and hospitals.

In still another embodiment the DBME comprises algorithms supported by experts in radiology from MRI subspecialties including head and neck, neuro and brain, bone, chest, abdomen, pelvis, breast and cardiac imaging.

In a further embodiment the DBME comprises algorithms supported by experts in radiology from PET/CT subspecialties including brain, chest, abdomen, pelvis, and cardiac imaging.

In still another embodiment the DBME comprises algorithms supported by experts in radiology from CT subspecialties including brain, head and neck, chest, abdomen, pelvis, and cardiac imaging.

In still another embodiment the DBME comprises algorithms supported by experts in radiology and internal medicine from SPECT/Nuclear Medicine diagnostic and therapeutic subspecialties.

In still another embodiment the DBME comprises algorithms supported by experts in cardiology, medical oncology, surgical oncology and radiation oncology.

In a further embodiment the DBME comprises algorithms supported by experts in cardiology having one or more subspecialties selected from pediatric cardiology, interventional cardiology, peripheral vascular, electrophysiology, and cardiac surgery.

In a further embodiment the DBME comprises algorithms supported by experts in medical oncology having one or more subspecialties selected from pediatric oncology, neuro-oncology, head and neck oncology, breast oncology, lung oncology, gastrointestinal oncology, gynecologic oncology and urologic oncology.

In a still further embodiment the DBME comprises algorithms supported by experts in surgical oncology having one or more subspecialties selected from pediatric surgical oncology, neurosurgical oncology, head and neck surgical oncology, breast surgical oncology, thoracic surgical oncology, abdominal surgical oncology, colorectal surgical oncology, gynecologic surgical oncology, and urologic surgical oncology.

In one embodiment the DBME comprises algorithms supported by experts in radiation oncology having one or more subspecialties selected from pediatric radiation oncology, neuro radiation oncology, head and neck radiation oncology, breast radiation oncology, lung radiation oncology, abdominal radiation oncology, colorectal radiation oncology, urologic radiation oncology, and gynecologic radiation oncology.

In another embodiment the physician accepts the diagnostic recommendation of the DBME or challenges the recommendation and provides deviations factors for further consideration by the DBME.

In a further embodiment the DRA comprises a detailed description of the medical and scientific basis of the recommendation.

Another embodiment provides a method further comprising: iv) providing notification from the physician to the DBME of the physician's acceptance of DRA recommendations.

Another embodiment provides a method further comprising processing CME credit award to the physician.

Another embodiment provides a method further comprising providing the physician with a certificate of credit for malpractice risk reduction.

Another embodiment provides a method further comprising v) providing notification from DBME to the physician of scheduled hi tech diagnostic study.

Another embodiment provides a method further comprising vi) providing notification from DBME to the Health Insurance Company of hi tech diagnostic study approval for payment.

Another embodiment provides a method further comprising vii) providing notification from DBME to the Medical Facility and viii) issuing clinical information, test schedule notification, and authorization for payment.

Another embodiment provides a method further comprising: ix) providing through a Patient Information Module (PIM) test schedule options available at the medical facility; and x) providing via PIM to medical facility patient appointment acceptance.

Another embodiment provides a method further comprising xi) providing test results from the medical facility to Digital Radiology Reading and Review Module (DRRM).

Another embodiment provides a method further comprising xii) providing test results and a reading of the test results from the medical facility to the Digital Radiology Reading and Review Module (DRRM).

Another embodiment provides a method further comprising xiii) checking through the DRRM the quality and comprehensiveness of the reading provided by the medical facility.

Another embodiment provides a method further comprising ixx) assessing through the DRRM the reading of the test results based on minimal subspecialty standards and accepting the reading or xx) forwarding through the DRRM the test results to a subspecialty expert for further reading.

Another embodiment provides a method further comprising xxi) authorizing through the DRRM payment to the facility of a global fee if the reading provided by the facility is acceptable or xxii) calculating and authorizing payment of a prorated fee if the reading was not accepted.

Another embodiment provides a method further comprising xxiii) processing through the TM the reading of the test results and determining whether a TRA is available for the findings, and if not forwarding the radiological readings to the physician without a therapeutic recommendation.

Another embodiment provides a method further comprising xxiv) processing through the TM the reading of the test results and determining whether a TRA is available for the findings, and if not forwarding to the physician a list of additional tests and/or results required to qualify for a TRA.

Another embodiment provides a method further comprising xxv) identifying through the TM a therapeutic algorithm for the findings and running the Therapeutic Recommendation Algorithm (TRA) to process the test results for determining treatment options.

Another embodiment provides a method further comprising xxvi) providing through the DBME treatment options to the physician based on test results.

In one embodiment the physician can accept treatment options or request consensus evaluation based on submitted deviation factors.

Another embodiment provides a method further comprising upon physician acceptance of treatment option, xxvii) recommending through the DBME to the physician a list of qualified facilities and specialists, and xxviii) expediting billing and automatic payment.

Another embodiment provides a method further comprising xxix) providing through the Patient Information Module the hi tech test recommendations in layman's terms; xxx) providing through the Patient Information Module the hi tech test results in layman's terms; xxxi) providing through the Patient Information Module accepted treatment options in layman's terms; and/or xxxii) providing through the Patient Information Module interactive tools to obtain informed consent from patient prior to acceptance of recommendations for diagnostic testing and or treatment.

Another embodiment provides a method further comprising xxxiii) providing notification from the physician to the DBME of hi tech diagnostic testing recommendations not accepted; and xxxiv) obtaining from the physician deviation factors for consensus review as submitted through the deviation factors submission algorithm (DFSA).

Another embodiment provides a method further comprising xxxv) submitting deviation factors for diagnostic consensus review algorithm (DCRA) by subspecialty experts; and xxxvi) conducting consensus evaluation by panel of sub-specialty experts for diagnostic options.

Another embodiment provides a method further comprising xxxvii conducting consensus vote:
(a) if majority of voting sub-specialists vote in favor of deviation factors then algorithm is modified and studies and/or treatments are approved;
(b) if majority vote is against deviation factors, then algorithm is unchanged and studies and/or treatment is denied;
(c) if vote is neutral (approximately half for and half against) then algorithm is unchanged but payment is approved due to lack of sub-specialty consensus (gray area of medical thought).

Another embodiment provides a method further comprising xxxiii) providing through the DBME results of consensus vote:
(a) if consensus vote is against deviation factors then recommendations by DRA are confirmed;
(b) If consensus vote accepts deviation factors then DRA is modified via diagnostic consensus improvement algorithm (DCIA) to include accepted deviation factors and recommendations are altered and approved for payment.

Another embodiment provides a method further comprising ixl) providing through Patient Information Module results of consensus review in layman's terms prepared through layman's terms conversion algorithm (LTCA).

Another embodiment provides a method further comprising xl) providing through the Patient Information Module physician deviation factors in layman's terms.

Another embodiment provides a method further comprising xli) providing notification from the physician to the DBME of non-acceptance of therapeutic recommendations and forwarding to DBME deviation factors entered by the physician; and xlii) submitting deviation factors for therapeutic consensus review through the therapeutic consensus review algorithm (TCRA) by subspecialty experts.

Another embodiment provides a method further comprising xliii) conducting consensus evaluation by a panel of sub specialty experts.

Another embodiment provides a method further comprising xliv) providing to the physician results of consensus vote:

(a) if consensus vote is against deviation factors then recommendations by TRA are confirmed; and

(b) if consensus vote accepts deviation factors then TRA is modified to include deviation factors and recommendations are altered and approved for payment.

Another embodiment provides a method further comprising xlv) providing through Patient Information Module results of consensus review in layman's terms.

Another embodiment provides a method further comprising xlvi) implementing modification of TRA via TCIA based on consensus vote.

Another embodiment provides a method further comprising xlvii) presenting to the patient information on relevant products and services relating to the patient's specific clinical situation selected through TAA including information compiled through DBME search engine and advertisers (pharmaceutical companies, equipment manufacturers, service providers).

Another embodiment provides a method further comprising xlviii) providing to the physician continuous medical educational (CME) credit through CMEM and sponsored by medical facilities, advertisers and/or health insurance companies to encourage physician participation.

Another embodiment provides a method further comprising il) providing to the physician reduced cost malpractice coverage through the MMRMM for following recommendations of the DBME.

Another embodiment provides a method further comprising l) processing payment to sub-specialty members of specific clinical algorithm board identified through the MM.

Another embodiment provides a method further comprising li) charging health insurance carrier for processing payment authorization for approval/denial of hi tech diagnostic test through the MM.

Another embodiment provides a method further comprising lii) crediting through PCPA patient's account for reduced deductible and/or co-pay based on patient acceptance of DBME diagnostic and/or therapeutic recommendations and also providing through the PCPA cost comparison information enabling patient to chose among qualified healthcare providers the lowest cost options.

Another embodiment provides a method further comprising liii) crediting through the MM the physician's account for submission of accepted deviation factors that resulted in modification of the specific diagnostic recommendation algorithm (DRA).

Another embodiment provides a method further comprising liv) processing through the MM payment to Sub-specialists participating in diagnostic consensus vote.

Another embodiment provides a method further comprising lv) crediting through the MM the physician's account for submission of accepted deviation factors that resulted in modification of the specific therapeutic recommendation algorithm (TRA).

Another embodiment provides a method further comprising lvi) processing through the MM payment to Sub-specialists participating in the diagnostic and therapeutic consensus review votes (DCRA, TCRA).

Another embodiment provides a method further comprising lvii) charging through the MM advertisers for ads placed on the Patient Information Module (PIM).

In one embodiment the ads comprise information on relevant products and services as relating to patient's specific clinical situation

Another embodiment provides a method further comprising lviii) charging through the MM advertisers for receiving relevant information from advertisers (pharmaceutical companies, equipment manufacturers, service providers) and loading the information on the patient information module.

In another embodiment the DBME charges through the MM advertisers based on information accessed by the patient through the PIM.

Another embodiment provides a method further comprising lix) charging through the MM advertisers for ads selected through PTAA and placed on Physician Action Module (PAM).

In another embodiment the ads comprise information on relevant products and services relating to the physician's specific clinical case.

Another embodiment provides a method further comprising lx) charging through the MM advertisers for receiving relevant information from advertisers (pharmaceutical companies, equipment manufacturers, service providers) and loading the information on the Physician Action Module (PAM).

In another embodiment the DBME charges through the MM advertisers based on information accessed by the physician through the PAM.

Another embodiment provides a method further comprising lxi) charging through the MM the malpractice insurance carrier for processing medical malpractice risk management credits for the physician.

Another embodiment provides a method further comprising lxii) charging through the MM the physician for billing the health insurance carrier for services rendered by physician in connection with ordering High Tech Diagnostic tests and/or providing treatment.

In one embodiment the services rendered by the physician comprise one or more of patient examination, CIS preparation and submission, ordering and evaluating test results and/or providing treatment.

Another embodiment provides a method further comprising lxiii) charging through the MM the medical facility for billing health insurance carrier for services rendered by the facility in connection with performing hi tech diagnostic tests and/or providing treatments.

Another embodiment provides a method further comprising charging through the MM the reading radiologist for billing health insurance carrier for services rendered by the reading radiologist in connection with reading the results of the hi tech Diagnostic test.

Another embodiment provides a method further comprising lxiv) charging through the MM the health insurance carrier for primary or overview radiological readings by qualified sub-specialists.

Another embodiment provides a method further comprising lxv) processing through the MM payment to sub-specialty members of specific clinical algorithm boards identified through TM.

Another embodiment provides a method further comprising lxvi) charging through the MM the health insurance carrier for processing payment authorization for approval/denial of treatment recommendation through TRA.

Another embodiment provides a method further comprising lxvii) charging through the MM the physician for billing the health insurance carrier for services rendered by the physician in connection with providing treatment.

Another embodiment provides a method further comprising lxviii) charging through the MM the medical facility for billing the health insurance carrier for services rendered by the facility in connection with performing the recommended treatments.

Another embodiment provides a method further comprising lxix) removing patient personal identifying information from clinical information by a de-coupler program and encryption identity assigned within the privacy compliance module (PCM).

Another embodiment provides a method further comprising lxx) removing physician personal identifying information from clinical information by a de-coupler program and encryption identity assigned within the privacy compliance module (PCM).

Another embodiment provides a method further comprising lxxi) Providing patient ID/Password Protection so that only patients can access their own information on the Patient Information Module (PIM) via a re-coupler program provided in the Privacy Compliance Module (PCM).

Another embodiment provides a method further comprising lxxii) providing physician ID/Password Protection so that only physicians can access their own patients information on the Physician Action Module (PAM) via a re-coupler program in the Privacy Compliance Module (PCM)

Another embodiment provides a method further comprising lxxiii) providing health insurance carrier ID/Password Protection so that only carriers can access their own patients subscriber information on the Insurance Authorization Module (IAM) via a re-coupler program in the Privacy Compliance Module (PCM).

Another embodiment provides a method further comprising lxxiv) providing health insurance carrier ID/Password Protection so that only carrier can access their own patients subscriber health care provider information on the Insurance Authorization Module (IAM) via a re-coupler program in the Privacy Compliance Module (PCM)

Another embodiment provides a method further comprising lxxv) providing medical facility ID/Password Protection so that only medical facility can access their own patients information on the Medical Facility Action Module (MFAM) via a re-coupler program in the Privacy Compliance Module (PCM).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B: FIG. 1A illustrates the first segment of the various modules and connections according to embodiments of the invention; FIG. 1B illustrates the second segment of the various modules and connections according to embodiments of the invention.

FIGS. 2A and 2B: FIG. 2A is the first segment of a flowchart illustrating a composition of a DBME according to one embodiment of the invention; FIG. 2B is the second segment of a flowchart illustrating a composition of a DBME according to one embodiment of the invention.

FIG. 3 is a simplified flow chart showing components of a platform according to one embodiment of the invention.

FIGS. 4A-1 to 4A-3, 4B-1 to 4B-2, 4C-1 to 4C-2 and 4D: FIG. 4A-1 is the first segment of a first flowchart illustrating a detailed description of the component of FIG. 3; FIG. 4A-2 is the second segment of a first flowchart illustrating a detailed description of the component of FIG. 3; and FIG. 4A-3 is the third segment of a first flowchart illustrating a detailed description of the component of FIG. 3. FIG. 4B-1 is the first segment of a second flowchart illustrating a detailed description of the component of FIG. 3; and FIG. 4B-2 is the second segment of a second flowchart illustrating a detailed description of the component of FIG. 3. FIG. 4C-1 is the first segment of a third flowchart illustrating a detailed description of the component of FIG. 3; and FIG. 4C-2 is the second segment of a third flowchart illustrating a detailed description of the component of FIG. 3. FIG. 4D is a fourth flowchart illustrating a detailed description of the component of FIG. 3.

FIGS. 5A to 5D: FIG. 5A is the first segment of a flowchart illustrating information flow according to one embodiment of the invention; FIG. 5B is the second segment of a flowchart illustrating information flow according to one embodiment of the invention; FIG. 5C is the third segment of a flowchart illustrating information flow according to one embodiment of the invention; and FIG. 5D is the fourth segment of a flowchart illustrating information flow according to one embodiment of the invention.

FIGS. 6A and 6B: FIG. 6A is the first segment of a flowchart illustrating privacy controls during information flow according to one embodiment of the invention; and FIG. 6B is the second segment of a flowchart illustrating privacy controls during information flow according to one embodiment of the invention.

FIGS. 7A to 7C: FIG. 7A is the first segment of a flowchart illustrating monetization of action items according to one embodiment of the invention; FIG. 7B is the second segment of a flowchart illustrating monetization of action items according to one embodiment of the invention; and FIG. 7C is the third segment of a flowchart illustrating monetization of action items according to one embodiment of the invention.

### DETAILED DESCRIPTION OF THE INVENTION

Embodiments of the present invention provide a method by which the cost of digitization of key medical information is absorbed by those healthcare stakeholders that economically profit from the application of the collected and applied medical information of the Digital Board of Medical Experts (DBME). For example, Health Insurance Carriers would pay for the processing of cases by the DBME in order to get financially unbiased recommendations on payment approvals or denials that are provided digitally by unbiased nationally recognized medical experts and less likely to be questioned or challenged by physicians and/or subscribers.

In the context of the present invention the phrase “providing a diagnostic and/or therapeutic recommendation” contemplates all avenues for communication the diagnostic and or therapeutic recommendations to the physician, patient, health insurance carrier, diagnostic or therapeutic facility or any other involved party. For example, and without limitation, the recommendations may be communicated to a party through printed document; email, posting on a website, posting on a portal, posting in secured area of a personal space, provided through intranet, saving the recommendation on a computer readable medium, etc.

Health Insurance Carriers currently pay for medical approval processing through a variety of mechanisms including in-house employed physicians whose medical impartiality is always questioned due to the common use of financial incentives that encourage medical denials.

Health Insurance Carriers would also pay for processing of the billing claims made by physicians and medical facilities who have received payment approvals for medical procedures approved by the DBME on behalf of the health insurance carriers. The Health Insurance Carriers costs for processing both payment approvals and billing claims is reduced due to efficiencies of shared data management through the DBME.

Also, physicians and medical facilities pay a fee for billing their claims to the Health Insurance Carriers for services provided that have been approved through the DBME. Billing costs and time elapsed before payment receipt is also reduced for physicians and medical facilities due to efficiencies of shared data management through the DBME.

Advertisers also help absorb the cost of digitization of medical information through the DBME, by paying advertising fees in order to be able to target highly selected populations of physicians, patients and medical facilities that are in highly motivated need of information on their products and/or services as identified through the data bases of the DBME.

The digital transfer of relevant and timely medical information is a key element to solving the problems of the U.S. healthcare system. This is particularly important in view of the fact that there is and continues to be an explosion of new medical information related to the diagnosis and treatment of life-threatening diseases thus making the high quality and efficient practice of medicine even more complex.

In addition, there is also an extensive and progressive proliferation of new and expensive high-end technology for radiological, laboratory and genetic testing requiring even greater expertise for proper utilization. If these high-end technologies are applied incorrectly, then that will lead to poor outcomes and even greater costs than the improper utilization of the less costly lower end technologies.

However, if properly applied, hi tech approaches can lead to greatly improved outcomes with less risk, complications and lower overall costs in the delivery of state of the art healthcare. Any inability for physicians to maintain a dynamic and current knowledge base will contribute greatly to poor clinical outcomes and consequentially increase the cost of healthcare. The present invention updates medical information on a daily basis.

The incorporation of clinical advances from a university hospital practice to a community practice takes over 5-10 years to gain status as a standard within the community. This is particularly troublesome in view of the fact that over 85% of all healthcare services are delivered within a community setting.

Concurrent with the explosion of new medical information and technology, there is an epidemic of documented medical errors with over 100,000 known hospital deaths per year and over 2 million ambulatory errors identified per year further confirming the complexities of high quality healthcare.

In order to address the problematic issues of the U.S. healthcare system in a practical manner, the present invention manages medical information efficiently and limits the accumulation of extraneous patient data by digitizing only the pertinent key elements in the crossroads of the medical decision-making process. Digitization of these key elements in the medical decision-making process permits the matching of a clinical index of suspicion to the most appropriate diagnostic and therapeutic procedures result in a significant impact on the subsequent level of quality and efficiency in the healthcare delivery process.

The data comprising the crossroads of the medical decision-making process is also most amenable to digitization because its key elements are finite by known clinical pathways and limited to the most current expert state of the art practices for a specific disease presentation.

The present invention provide flexible tool which allow tailoring practical approaches applied to selected disease presentations that have a high probability for better outcomes with lower costs. The tools of the present invention, designed to be used by all physicians from general practitioners to specialists, will be applied initially to clinical presentations requiring expertise in the sub-specialty fields of radiology, cardiology and oncology and then expanded top other high cost area of healthcare.

The present invention through the internal and external core modules of the DBME manages essential information that directly impacts the critical efficiency and quality needs of patients, physicians, medical facilities and health insurance carriers.

Patients would like to have relevant and highly specific information about their own medical condition that would help them not only understand their specific disease process but also help them find the “best doctor” or more accurately confirm the knowledgeably of their chosen physician for their particular medical problem. In addition, with the promotion of health insurance accounts yielding lower premiums but higher deductibles and co-pays, patients will be called upon to make more knowledgeable decisions regarding healthcare choices and will be even more in need of relevant and understandable medical information that is specifically applicable to their medical situation.

Patients that attempt searches on the internet through websites such as WebMD or through the popular search engines or pharmaceutical companies or medical device manufacturers are usually inundated with either biased incomplete information and/or massive amounts of bewildering information presented in a format of equal importance that in most cases are irrelevant to their specific medical situation. Therefore, the information obtained is mostly useless in helping patients make an informed decision on their own specific medical condition. The present invention corrects this situation by providing medical information to the patient through the DBME that has been highly selected out by their own physicians' data input (clinical index of suspicion). The patient's own physician is in the best position to narrow the information search for specific relevancy to the patient. It is the same DBME analyzed medical information that is being used by their own physicians to determine diagnostic and therapeutic procedures. The medical information, however, has been translated into layman's terms for ease of understanding by the patient. The present invention also provides the patient with an optimized informed consent due to the transparent nature of the DBME information generated and converted into layman's terms through the layman's terms conversion algorithm (LTCA).

It is the patient's fervent hope and desire that their chosen physician is making decisions based on the latest medical information and that his or her knowledge is on par with the most knowledgeable physicians in the country and even the world. It is for this reason that most patients, once confronted with a life-threatening diagnosis, embark on a desperate quest for the “best physician”. However, depending on their personal resources, their quest may be limited by lack of relevant information. It is generally believed that not all physicians have the same knowledge and skills as all other physicians. Therefore, patients make decisions in finding “the best physician” based on affiliations with major institutions, recent publicity and notoriety, and personal recommendations from friends or relatives. None of these methods can guarantee that the patient has found a physician with state of the art knowledge for the disease for which the patient presents. In addition, if patients could accurately identify the most knowledgeable physicians for a particular disease presentation, then these physicians would not be able to handle the sear volume of patients that present to them for diagnosis and therapy.

As a practical matter, there just aren't enough expert sub-specialists (specialists who limit their expertise to an intensive mastering of a sub-category of disease within a particular specialty) to personally treat each patient with the sub-category of disease in which they sub-specialize.

Therefore, embodiments of the present invention provide an efficient solution for patients by creating a method by which all patients can be assured in a verified and completely transparent manner that their chosen physicians have access to and employ the same expert knowledge of the sub-specialists through a digital link (Digital Board of Medical Experts) that verifies and compares the physician's current knowledge base to that of the sub-specialists consensus knowledge base as the physician goes through the critical decision making process in real time for ordering hi tech diagnostic studies and/or selecting or delivering treatments.

Embodiments of the present invention provide patients with credits against medical insurance co-pays and/or deductibles when they agree to diagnostic and/or therapeutic regimens that are approved by their physicians and also recommended by the DBME. In addition, patients are provided with co-pay/deductible cost comparisons in order to identify qualified healthcare providers that are also the most cost effective for the patient.

The present invention also provides the patient with an optimized informed consent due to the transparent nature of the DBME information generated and converted into layman's terms through the layman's terms conversion algorithm (LTCA).

Embodiments of the present invention provide the patient with automatic health insurance payment approvals for medical services reviewed through the DBME thereby saving the patient time and extra effort by eliminating the need for obtaining referrals and insurance authorizations and reducing paperwork for the patient.

Embodiments of the present invention provide the patient with full anonymity to both identity and locale via the Privacy Compliance Module (PCM) while providing automatic scheduling of appointments.

Embodiments of the present invention provide for the patient to receive relevant advertising information directly related to their immediate healthcare interests via the targeted advertising algorithm (TAA) for patients while maintaining full anonymity via the Privacy Compliance Module (PCM).

Physicians would like to have the latest and most accurate medical information presented in a practical and useful manner that would enhance their clinical productivity for specific cases and also shield them from malpractice claims.

However, all physicians make diagnostic and therapeutic decisions based on an educational bias and also, in some cases, a financial bias whereby physicians may not even be aware that their knowledge base has been undermined by a financial bias toward certain diagnostic or therapeutic practices. An educational bias is developed based on the information that the physician has mastered throughout his medical school education, post-graduate residency training and continuing medical education acquired while in practice.

Some physicians are more diligent than others in maintaining an up-to-date knowledge base. This is, however, difficult in view of the constant developments in the dynamic environment of healthcare innovation with thousands of medical articles published monthly. Many physicians have mountains of unread journal articles piled high in their offices or next to their easy chairs at home for “must” reading that never happens because they become overwhelmed.

Although physicians have an obligation to seek unbiased sources of medical education, drug companies and equipment manufactures routinely present information through a sales force of detail representatives (as known as detail men or women) that disperse samples and call attention to medical articles that present a favorable yet biased opinion on products that they are marketing. All too often these biased presentations are accepted as equivalent to independent medical information.

Patients do not know whether their physician is making a recommendation based on the latest and most effective medical information or whether it is based on stale and/or one-sided and incomplete information.

Likewise, patients do not know whether recommendations for diagnostic or therapeutic procedures are based on some financial bias due to various incentives to which the physician is subjected either overtly or subtly. It is human nature to prefer methods that create greater financial benefit and physicians are only human.

The unquestionable influence on physicians of incomplete and biased medical information presented by representatives of drug and medical device companies has led to the banning of detail men and women at some major medical institutions. Yet, the vast majority of diagnostic and therapeutic care is delivered in the community setting where such bans are essentially non existent because “detail presentations” are viewed as providing a convenient source of new although biased medical information to physicians. There is a general belief by physicians that they can mentally filter out the biased elements of drug company presentations but that has not been the experience of the drug and equipment manufactures that go to great lengths to influence physicians. Companies with weaker products often create greater financial benefits for physicians to overcome any competitive weaknesses in their products.

Embodiments of the present invention provide a practical solution with a seamless digital integration of the physician work process that saves the physician time and patient processing expense while updating the physician with the most recent and effective information for the specific case at hand as the physician proceeds to manage the case.

Embodiments of the present invention provide a method for both physician and locale anonymity while providing a “best practices” educational review on a case by case basis with the physician receiving CME credits for interaction with DBME via the Continuous Medical Education Module (CMEM) thus enabling physicians to maintain up-to-date knowledge of clinical advances that are immediately relevant to their specific cases as part of the normal physician work process thus eliminating physician education bias influenced by previous experiences or previous training which has not been updated.

The present invention streamlines and standardizes the physician decision making process regarding the proper utilization of hi tech diagnostics and therapeutics via the diagnostic recommendation algorithm (DRA) and/or the therapeutic recommendation algorithm (TRA) thus eliminating referring physician peer pressure to select diagnostic and/or therapeutic options that are biased by the referring physician and eliminating financial bias in the diagnostic and/or therapeutic selection process.

Embodiments of the present invention provide a method which qualifies the physician for a malpractice insurance discount or credit against his malpractice insurance premium with the physician's concurrence or acceptance of the DBME recommendation via the Medical Malpractice Risk Management Module (MMRMM).

Embodiments of the present invention provide a digital record of diagnostic findings and/or therapeutic recommendations which assures timely intervention based on the digital findings via the diagnostic time sensitive response algorithm (DTSRA) or the therapeutic time sensitive response algorithm (TTSRA) with online notification of report availability.

Embodiments of the present invention provide for health insurance payment approval for automatic referral payment and automatic physician payment which saves cost and personnel processing time for billing and also for automatic scheduling at the most appropriate facility with qualified and matched capabilities to the clinical situation.

Embodiments of the present invention provide for the elimination of the time consuming need for physician advocacy when seeking payment authorization for new cutting edge diagnostics or therapies that are usually rejected by health insurance carriers as “experimental” when in fact they are state of the art advances thus inhibiting the swift adoption of more effective newer therapies. Diagnostic and/or therapeutic recommendations through the DBME are automatically accepted for payment because these recommendations are derived from the recognized and accepted experts in the field.

Embodiments of the present invention provide for the digitization and analysis of a patient's clinical index of suspicion observed by the physician and processed via the clinical index of suspicion selection algorithm (CISSA) which digitizes elements such as patient's complaints, symptoms, physical exam findings, lab and radiological test results and other findings enabling a digital analysis of the physician provided clinical index of suspicion for confirmation of the most appropriate hi tech testing and/or therapeutic recommendation.

Embodiments of the present invention provide for the further personalization of the diagnostic and/or recommendation process by providing a method via the deviation factors submission algorithm (DFSA) that allows for adjustment analysis of physician provided deviation factors that may impact testing and treatment appropriateness of the recommended algorithm. This assures that all algorithm recommendations are personalized to any specific and unique factors of the patient's case not anticipated by the recommendation algorithm.

Embodiments of the present invention provide for a DBME consensus evaluation of submitted deviation factors by the physician via the diagnostic consensus review algorithm (DCRA) or the therapeutic consensus review algorithm (TCRA). Consensus affirmation by experts of submitted deviation factors results in modification of the recommendation algorithm assuring continuous improvement of the diagnostic and therapeutic algorithm via the diagnostic consensus improvement algorithm (DCIA) or therapeutic consensus improvement algorithm (TCIA). The present invention further provides a method by which the physician receives compensation for submitting successful algorithm-modifying deviation factors.

Embodiments of the present invention provide for a method by which the physician receives a referral list of local qualified specialists who accept the therapeutic recommendations of the DBME via the therapeutic specialist qualification & assignment algorithm (TSQAA) which saves time for the physician in identifying qualified local specialists who would treat the patient according to the DBME recommendation.

Embodiments of the present invention provide a method by which the physician receives relevant advertising information related to the clinical needs of his/her practice via the physician targeted advertising algorithm (PTAA).

Medical facilities would like to have widespread dissemination of information regarding the unique features of their hi tech equipment that would assure referrals and justify large capital investments in the latest technology. However, both physicians and the lay public alike may not have an accurate understanding of the characteristics and capabilities of hi tech diagnostic and therapeutic equipment that is located in their community. The present invention through the Medical Facility Action Module (MFAM) provides a method by which medical facilities can have their technological capabilities correctly matched to the clinical needs of patients for diagnostic and/or therapeutic services.

Embodiments of the present invention provide for proper utilization of hi tech equipment by algorithm matching tracked by the equipment utilization algorithm (EUA) of the MFAM thereby eliminating any educational and/or financial biases of referring physicians.

Embodiments of the present invention provide a method for reduced radiological reading fees due to efficient expert digital readings by DBME subspecialty radiologists through the Digital Radiology Reading & Review Module (DRRM). Whereas, local radiologists with proper subspecialty credentials can qualify to be digital readers via the specialist qualification algorithm (SQA) of the MFAM.

Embodiments of the present invention provide a method by which hospitals and independent centers can participate by registering their existing hi tech equipment into a digitally profiled equipment database via the facility & personnel qualification algorithm (FPQA) of the MFAM which also confirms technician capability of the facility.

Embodiments of the present invention provide a method for automatic scheduling at participating medical facilities via the scheduling algorithm (SA) of the MFAM.

Embodiments of the present invention provide a method by which medical facilities can have automatic payment authorization and automatic payment via the payment approval algorithm (PAA) of the Health Insurance Authorization Module (IAM) and reduction of billing costs for medical facilities via the billing algorithm (BA) of the IAM.

Health insurance carriers would like to have relevant and indisputable information that would permit cost-effective evaluation methods for appropriate payment approvals or denials. There are significant regional and even community variations in the practice of medicine making it difficult to properly assess the medical appropriateness of hi tech diagnostic and therapeutic procedures.

Health insurance companies have been routinely criticized for employing medical personnel that have a financial incentive in claims denial to determine medical appropriateness as a basis for approving or denying payment authorization for healthcare services. Congressional testimony at investigative committees has confirmed that physicians employed by the health insurance companies have been encouraged and even coerced through financial incentives to deny payment authorization for expensive hi tech diagnostic and therapeutic procedures. Nevertheless, insurance companies do have the right to deny payment for inappropriate therapies and to guard against fraud and abuse.

Embodiments of the present invention provide a method whereby payment approval is based on DBME “best practices” confirmation by recognized experts in their fields through the payment approval algorithm (PAA) of the IAM via the diagnostic recommendation algorithm (DRA), the diagnostic consensus review algorithm (DCRA), therapeutic recommendation algorithm (TRA) and the therapeutic consensus review algorithm (TCRA).

Embodiments of the present invention provide a method whereby conflict of interest criticisms for health insurance companies can be eliminated by removing payment authorization from financially incentive-based employed physicians and nurses and placing the diagnostic and therapeutic approval process in the hands of impartial nationally recognized sub-specialty experts through the DBME.

Embodiments of the present invention provide a method which lowers the cost of healthcare services by denying payment authorization for ineffective, unnecessary, inefficient, outdated and unnecessarily risky procedures as determined by nationally recognized subspecialty experts of the DBME and eliminates the cost for health insurance companies of “blind” compliance audits since every hi tech diagnostic and/or therapeutic procedure recommended by the DBME is compliant with “best practices”.

Embodiments of the present invention provide a method which eliminates fraud and abuse concerns for health insurance companies regarding financially based conflict of interest referrals for unnecessary diagnostic or therapeutic services.

Embodiments of the present invention provide a method that lowers the cost of processing healthcare provider claims and payments through the digitally integrated system of the DBME via the payment approval algorithm (PAA) and the billing algorithm (BA) of the IAM and allows for differential cost adjustments with higher deductibles and co-pays for patients who do not follow DBME recommendations.

It is currently estimated that as much as half of the high cost hi tech diagnostic or therapeutic procedures are either underutilized, over-utilized or misused resulting in costly inefficiencies throughout the healthcare system. The utilization of expert opinion confirmation for all high cost and complex hi tech diagnostics and therapeutics through the DBME as standard practice will result in unprecedented cost efficiencies related to better outcomes with fewer complications and less risk for the patient. It is estimated that savings of up to 20% of total medical costs in the United States can be achieved if updated expert information is digitally incorporated into the diagnosis and treatment of each patient. Today, it is estimated that the annual healthcare costs in the United States are approaching 2.6 trillion dollars by 2010. A savings of 20% would result in approximately half a trillion dollars becoming available for the improvement of the economy and the standard of care and/or medical coverage provided to the US population. As well, increasing the efficiency and reducing the cost of healthcare may be critical for saving the healthcare system from collapse as predicted by many in the industry.

Embodiments of the present invention provide a computerized method of managing medical care through communication between a Digital Board of Medical Experts (DBME), a physician; a health insurance carrier; one or more medical facilities; and a patient and providing a diagnostic and/or therapeutic recommendation, the method comprising the following steps: i) providing a DBME core comprised of modules and algorithms for processing medical data and providing diagnostic and/or therapeutic recommendations; ii) providing a Physician Action Module (PAM) whereby a physician provides a clinical index of suspicion (CIS) through a clinical index of suspicion selection algorithm (CISSA) of the PAM to the DBME—for obtaining a recommendation for hi tech diagnostic studies and/or treatment; and iii) processing through the DBME information from the CISSA and providing a diagnostic recommendation from the diagnostic recommendation algorithm (DRA) of the Diagnostic Module (DM) of the DBME for hi tech diagnostic studies and/or a therapeutic recommendation from the therapeutic recommendation algorithm (TRA) of the Therapeutic Module (TM) of the DBME for treatment.

In one embodiment, the DBME comprises one or more of a diagnostic module (DM), a therapeutic module (TM), a digital radiological reading and review module (DRRM), a patient information module (PIM), a physician action module (PAM), an insurance authorization module (IAM), a medical facility action module (MFAM), a continuous medical education module (CMEM), a monetization module (MM), a medical malpractice risk management module (MMRMM), and a privacy compliance module (PCM).

In another embodiment, the PAM comprises a clinical index of suspicion selection algorithm (CISSA), a deviation factors submission algorithm (DFSA), a therapeutic specialist qualification and assignment algorithm (TSQAA), and a physician targeted advertising algorithm (PTAA).

Another embodiment provides a method further comprising selection by the physician of a CIS from a CIS menu processed through the CISSA.

In another embodiment, the PIM comprises a layman's terms conversion algorithm (LTCA), a targeted advertising algorithm for patients (TAA), and a patient's credit processing and cost comparison for co-pay/deductible algorithm (PCPA).

Yet another embodiment provides a method wherein the DM comprises one or more of a diagnostic recommendation algorithm (DRA), diagnostic consensus review algorithm (DCRA), a diagnostic consensus improvement algorithm (DCIA), and a diagnostic time sensitive response algorithm (DTSRA).

In another embodiment, the TM comprises one or more of a therapeutic recommendation algorithm (TRA), a therapeutic consensus review algorithm (TCRA), a therapeutic consensus improvement algorithm (TCIA), a prevention recommendation algorithm (PRA), and a therapeutic time sensitive response algorithm (TTSRA).

In still another embodiment the MFAM comprises one or more of a facility and personnel qualification algorithm (FPQA), a specialist qualification algorithm (SQA), a scheduling algorithm (SA), and an equipment utilization algorithm (EUA).

In still another embodiment the DRRM comprises one or more of a test quality assessment algorithm (TQAA), a test reading quality assessment algorithm (TRQAA), a payment proration algorithm (PPA); a performance tracking algorithm (PTA); and a reading specialist qualification and assignment algorithm (RSQAA).

In still another embodiment, the IAM comprises a payment approval algorithm (PAA) and/or a billing algorithm (BA).

In one embodiment the DRA comprises determining an overlay fidelity index (OFI) between the CIS provided by the physician and DBME defined factors.

In another embodiment the overlay fidelity index required by the DRA is determined based on the type of test, the invasiveness of the test, risk to the patient associated with the test and cost of the test.

In still another embodiment the DM and TM of the DBME are formed by recommendation algorithms (DRA, TRA, PRA) developed by experts from major Universities and Clinics, who are thought leaders and recognized in their sub-specialty fields of medicine who are identified and chosen through the ABSSM. In one embodiment the experts are organized in DCRA, DRA, TRA, TCRA and RSQAA boards. In a further embodiment the algorithms are digitally linked for immediate access for diagnostic and therapeutic evaluations and recommendations. In still another embodiment the DBME comprises algorithms supported by a board of experts grouped according to a set of subspecialties. In still another embodiment the experts are selected through the ABSSM based on their publications, lectures, clinical experience, faculty affiliations, positions within medical specialty colleges and associations, government agencies, national and international bodies, foundations, clinics and hospitals.

In a still further embodiment the DBME comprises algorithms supported by experts in surgical oncology having one or more subspecialties selected from pediatric surgical oncology, neuro-surgical oncology, head and neck surgical oncology, breast surgical oncology, thoracic surgical oncology, abdominal surgical oncology, colorectal surgical oncology, gynecologic surgical oncology, and urologic surgical oncology.

In another embodiment the physician accepts the diagnostic recommendation of the DBME or challenges the recommendation and provides deviations factors for further consideration by the DBME.

Another embodiment provides a method further comprising xxv) identifying through the TM a therapeutic algorithm for the findings and running the therapeutic recommendation algorithm (TRA) to process the test results for determining treatment options.

Another embodiment provides a method further comprising xxvi) providing through the DBME treatment options to the physician based on test results.

In one embodiment the physician can accept treatment options or request consensus evaluation.

Another embodiment provides a method further comprising xxxv) submitting deviation factors for diagnostic consensus review algorithm (DCRA) by subspecialty experts; and xxxvi) conducting consensus evaluation by panel of sub specialty experts for diagnostic options.

Another embodiment provides a method further comprising xxxvii conducting consensus vote:

(a) if majority of voting sub-specialists vote in favor of deviation factors then algorithm is modified and studies and/or treatments are approved;

(b) if majority vote is against deviation factors, then algorithm is unchanged and studies and/or treatment is denied;

(c) if vote is neutral (approximately half for and half against) then algorithm is unchanged but payment is approved due to lack of sub-specialty consensus (gray area of medical thought).

Another embodiment provides a method further comprising xxxiii) providing through the DBME results of consensus vote:

(a) if consensus vote is against deviation factors then recommendations by DRA are confirmed;

(b) If consensus vote accepts deviation factors then DRA is modified via diagnostic consensus improvement algorithm (DCIA) to include deviation factors and recommendations are altered and approved for payment.

Another embodiment provides a method further comprising xli) providing notification from the physician to the DBME of non-acceptance of therapeutic recommendations and forwarding to DBME deviation factors entered by the physician; and xlii) submitting deviation factors for therapeutic consensus review.

Another embodiment provides a method further comprising xliii) conducting consensus evaluation by a panel of sub specialty experts.

Another embodiment provides a method further comprising xliv) providing to the physician results of consensus vote:

(a) if consensus vote is against deviation factors then recommendations by TRA are confirmed; and

(b) if consensus vote accepts deviation factors then TRA is modified to include deviation factors and recommendations are altered and approved for payment.

Another embodiment provides a method further comprising xlv) providing through Patient Information Module (PIM) results of consensus review in layman's terms.

Another embodiment provides a method further comprising xlvi) implementing modification of TRA via TCIA based on consensus vote.

Another embodiment provides a method further comprising xlvii) presenting to the patient information on relevant products and services relating to the patient's specific clinical situation selected through TAA of the PIM including information compiled through DBME search engine and advertisers (pharmaceutical companies, equipment manufacturers, service providers).

Another embodiment provides a method further comprising xlviii) providing to the physician continuous medical educational credit (CME) through CMEM and sponsored by medical facilities, advertisers and/or health insurance companies to encourage physician participation.

Another embodiment provides a method further comprising il) providing to the physician reduced cost malpractice coverage through the MMRMM as credits against their medical malpractice insurance premium for following recommendations of the DBME.

Another embodiment provides a method further comprising l) processing payment to sub-specialty members of specific clinical algorithm board identified through the MM.

Another embodiment provides a method further comprising li) charging health insurance carrier for processing payment authorization for approval/denial of hi tech diagnostic test through the MM.

Another embodiment provides a method further comprising lii) crediting through PCPA patient's account for reduced deductible and/or co-pay based on patient acceptance of DBME diagnostic and/or therapeutic recommendations and also providing through the PCPA cost comparison information enabling patient to chose among qualified healthcare providers the lowest cost options.

Another embodiment provides a method further comprising liii) crediting through the MM the physician's account for submission of accepted deviation factors that resulted in modification of the specific diagnostic recommendation algorithm (DRA).

Another embodiment provides a method further comprising liv) processing through the MM payment to Sub-specialists participating in diagnostic consensus vote.

Another embodiment provides a method further comprising lv) crediting through the MM the physician's account for submission of accepted deviation factors that resulted in modification of the specific therapeutic recommendation algorithm (TRA).

Another embodiment provides a method further comprising lvi) processing through the MM payment to Sub-specialists participating in the diagnostic and therapeutic consensus review votes (DCRA, TCRA).

Another embodiment provides a method further comprising lvii) charging through the MM advertisers for ads placed on Patient Information Module (PIM).

In one embodiment the ads comprise information on relevant products and services as relating to patient's specific clinical situation.

Another embodiment provides a method further comprising lviii) charging through the MM advertisers for receiving relevant information from advertisers (pharmaceutical companies, equipment manufacturers, service providers) and loading the information on the patient information module.

In another embodiment the DBME charges through the MM advertisers based on information accessed by the patient through the PIM.

Another embodiment provides a method further comprising lix) charging through the MM advertisers for ads selected through PTAA and placed on Physician Action Module (PAM).

In another embodiment the ads comprise information on relevant products and services relating to the physician's specific clinical case.

Another embodiment provides a method further comprising lx) charging through the MM advertisers for receiving relevant information from advertisers (pharmaceutical companies, equipment manufacturers, service providers) and loading the information on the Physician Action Module (PAM).

In another embodiment the DBME charges through the MM advertisers based on information accessed by the physician through the PAM.

Another embodiment provides a method further comprising lxi) charging through the MM the malpractice insurance carrier for processing medical malpractice risk management credits for the physician.

Another embodiment provides a method further comprising lxii) charging through the MM the physician for billing the health insurance carrier for services rendered by physician in connection with ordering high tech diagnostic tests and/or providing treatment.

In one embodiment the services rendered by the physician comprise one or more of patient examination, CIS preparation and submission, ordering and evaluating test results and/or providing treatment.

Another embodiment provides a method further comprising lxiii) charging through the MM the medical facility for billing health insurance carrier for services rendered by the facility in connection with performing high tech diagnostic tests and/or providing treatments.

Another embodiment provides a method further comprising charging through the MM the reading radiologist for billing health insurance carrier for services rendered by the reading radiologist in connection with reading the results of the high tech diagnostic test.

Another embodiment provides a method further comprising lxiv) charging through the MM the health insurance carrier for primary or overview radiological readings by qualified sub-specialists.

Another embodiment provides a method further comprising lxv) processing through the MM payment to sub-specialty members of specific clinical algorithm boards identified through TM.

Another embodiment provides a method further comprising lxvi) charging through the MM the health insurance carrier for processing payment authorization for approval/denial of treatment recommendation through TRA.

Another embodiment provides a method further comprising lxvii) charging through the MM the physician for billing the health insurance carrier for services rendered by the physician in connection with providing treatment.

Another embodiment provides a method further comprising lxviii) charging through the MM the medical facility for billing the health insurance carrier for services rendered by the facility in connection with performing the recommended treatments.

Another embodiment provides a method further comprising lxix) removing patient personal identifying information from clinical information by a de-coupler program and encryption identity assigned within the Privacy Compliance Module (PCM).

Another embodiment provides a method further comprising lxx) Removing physician personal identifying information from clinical information by a de-coupler program and encryption identity assigned within the Privacy Compliance Module (PCM).

Embodiments of the present invention provide a platform for digital medicine based on a digital board of medical experts (DBME). The platform is fully secured and patient information remains confidential at all times.

The DBME has fully automated components as well as direct contact components whereby members of the DBME are called upon to evaluate a particular situation. The automated aspects of the DBME are based on algorithms built for each subspecialty for diagnosis, therapy and follow-up proposals to a doctor and a patient. The algorithms incorporate input from highly qualified and recognized members of each subspecialty. It is contemplated that most physician requests will be processed fully automatically.

In one aspect, the decision making process incorporated in the algorithms allows for automatic pre-approval of particular tests or therapy for reimbursement by an insurance carrier.

The DBME platform of the invention is advantageous in that it provides a streamlined process for selecting the most cost effective diagnostic tests and therapy regimens based on algorithms developed by highly recognized experts.

Accordingly, the invention provides a highly automated system that would allow the physician to obtain a recommendation based on a preliminary evaluation of the patient and an analysis conducted through an algorithm prepared and maintained by highly recognized experts in a particular subspecialty.

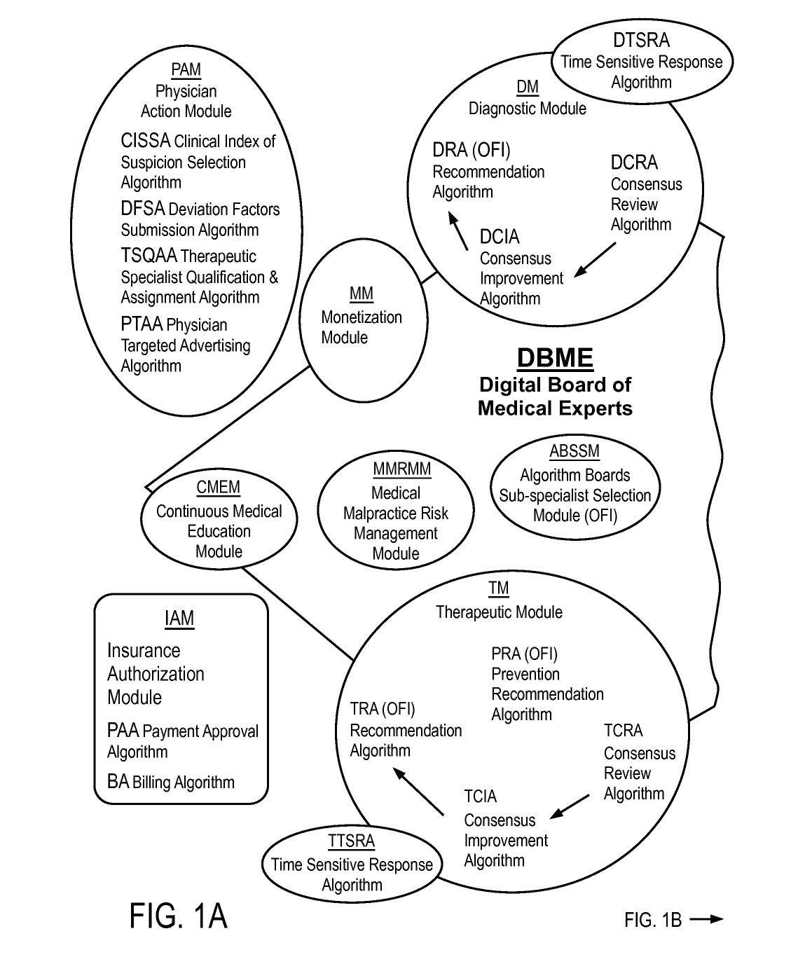

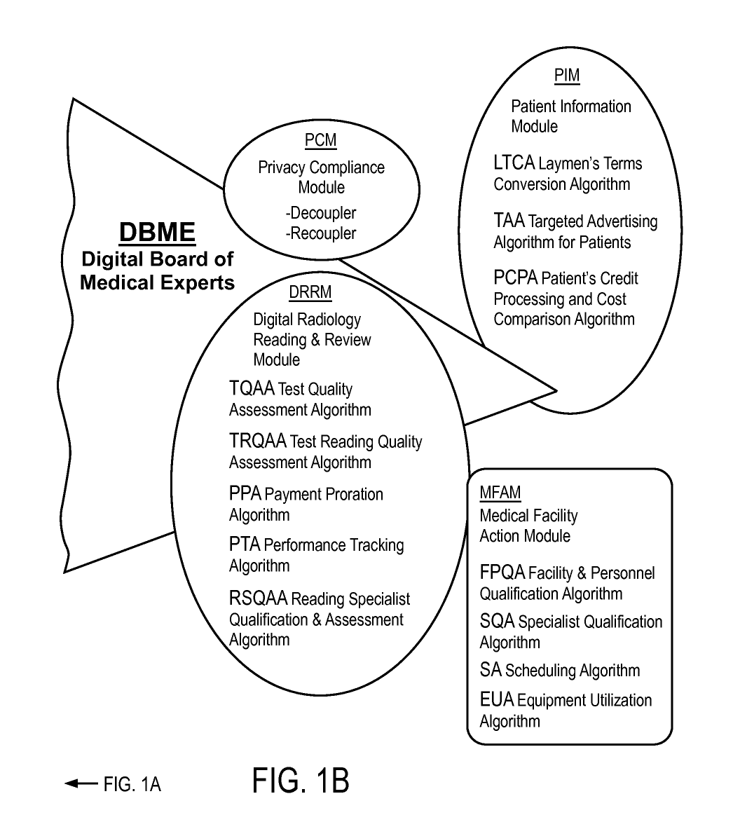

FIG. 1 summarizes the various components that form the DBME platform. It should be understood however that the invention does not require all the components in order for the DBME to deliver efficient diagnostic and/or therapeutic recommendations and assessments. The invention contemplates various subsets of the capabilities illustrated in FIG. 1.

As shown in FIG. 1, one embodiment provides a DBME with an interface for a physician to connect with DBME. This physician interface allows the physician to submit a preliminary patient evaluation through the CISSA of the PAM. For example, the physician may enter description of observations based on a physical examination of the patient and/or interview of the patient. The preliminary diagnostic information may also include results from routine tests such as simple blood tests or x-ray examinations. The preliminary evaluation is generally sufficient for the physician to suspect a particular disease or ailment. The CISSA assists the physician in entering the appropriate CIS data necessary for a DRA or TRA recommendation.

For example, if the physician is considering a high cost diagnostic test, the physician submits the patient information to the DBME through a physician interface. An analysis of the information provided by the physician through the CISSA is conducted and matched by an overlay fidelity index (OFI) to the most appropriate DRA which is selected to confirm whether a proposed diagnostic test is proper for the particular patient in that particular clinical situation.

If the analysis of the patient information results in a positive recommendation by the DBME, a number of actions are initiated. A space is created for the transaction and a report that DBME validated the request for the diagnostic test is sent to the requesting physician. In the report, information is provided for the physician to transmit to the patient so that the patient can access space dedicated to this transaction on the DBME system. Alternatively; the system may send an email directly to the patient indicating that a test has been approved. The system then locates a qualified facility that is convenient for the patient to conduct the test. Once the patient has selected a facility from one or more facilities identified by the system, the facility is electronically contacted and contact is established between the patient and the facility for scheduling a visit by the patient to the facility to conduct the test. The report sent to the physician optionally contains a pre-approval code which allows the physician to automatically submit the costs of the test for reimbursement by an insurance carrier.

The system also selects an expert for reading the results of the test. Once the test is conducted the results are digitized and forwarded to an expert for reading the results of the test. The expert prepares a report and sends the report electronically to the treating physician. A short summary of the test results written in simple words that can be understood by a lay person is sent to the patient.

Once the test results have been reviewed by the treating physician, the physician prepares a final diagnosis and initiates a new round of communications with the DBME. A therapeutic algorithm is selected based on the diagnosis information provided by the physician and the high tech test conducted through the DBME. The DBME prepares and forwards a therapeutic recommendation to the physician. The report containing the therapeutic recommendation also contains a pre-approval code which facilitates processing of the treatment costs by the insurance carrier. If appropriate, a facility for carrying out the treatment is identified and the patient is introduced to the facility so that scheduling of treatment sessions is facilitated.

If the algorithm analysis determines that the diagnostic test proposed by the physician is not proper, a report denying the test is sent to the physician. The report may include an alternative recommendation for another test. The report may also include a recommendation that other avenues be pursued before recommending the high tech diagnostic test. If the physician accepts the recommendations of the DBME, the physician can proceed based on the alternative diagnosis approaches contained in the report.

If the physician disagrees with the recommendations reached based on the algorithm analysis, the physician is invited to submit deviation factors. The deviation factors are sent electronically to selected members of the DBME appropriate for the case being reviewed as identified by the ABSSM. Each member of the DBME considers the deviation factors proposed by the treating physician. Each DBME member then electronically votes as to whether the deviation factors are acceptable or not. If a majority of the DBME members vote for accepting the deviation factors, the high tech diagnostic test is approved and the process described above is followed so that the patient is tested and potential therapeutic recommendations are made. In addition, if the deviation factors are accepted, the particular algorithm employed is revised to incorporate the deviation factors.

If a majority of the DBME members vote to deny the deviation factors, then the denial of the test is confirmed and a report is sent to the physician, to the patient and to the insurance carrier to reflect the decision of the DBME.

In situations where only a small majority of the DBME members, for example between 50% and 60% vote to deny the test, then the particular test may be approved. In this case the algorithms are not revised to include the deviation factors proposed by the treating physician.

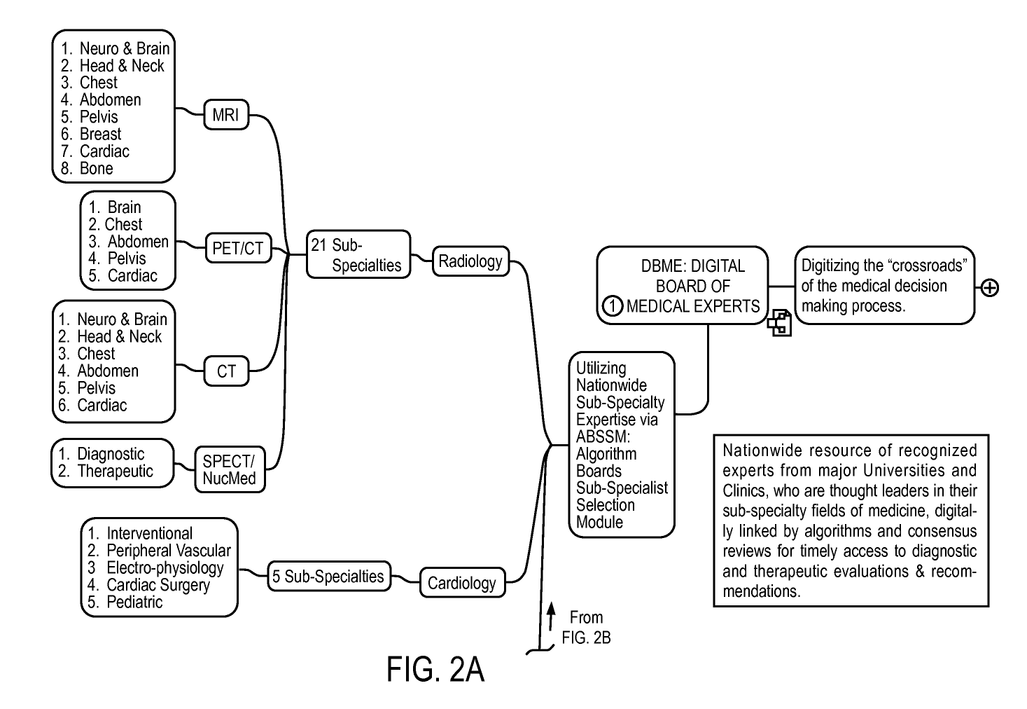

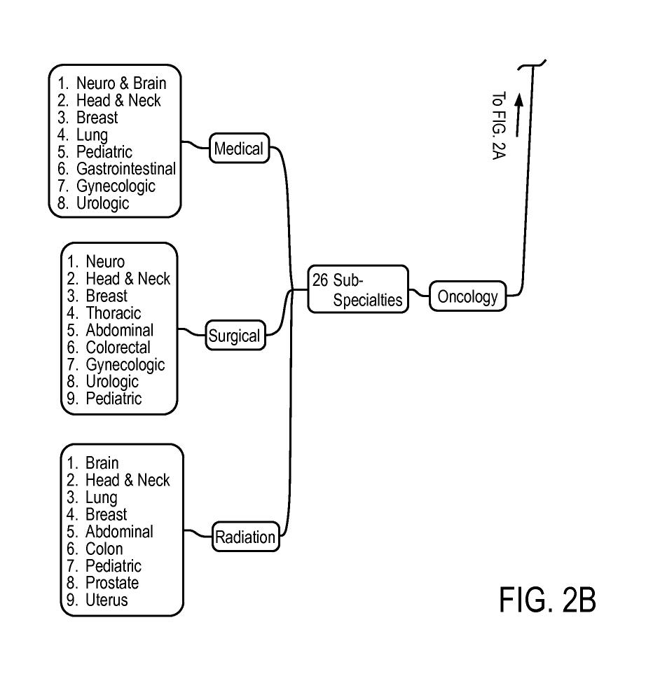

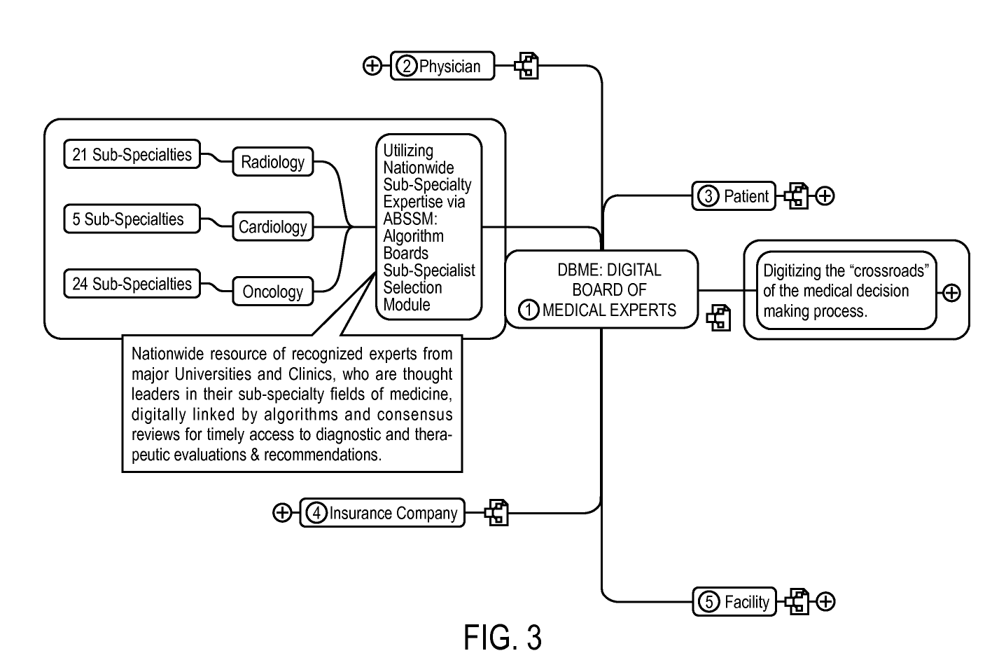

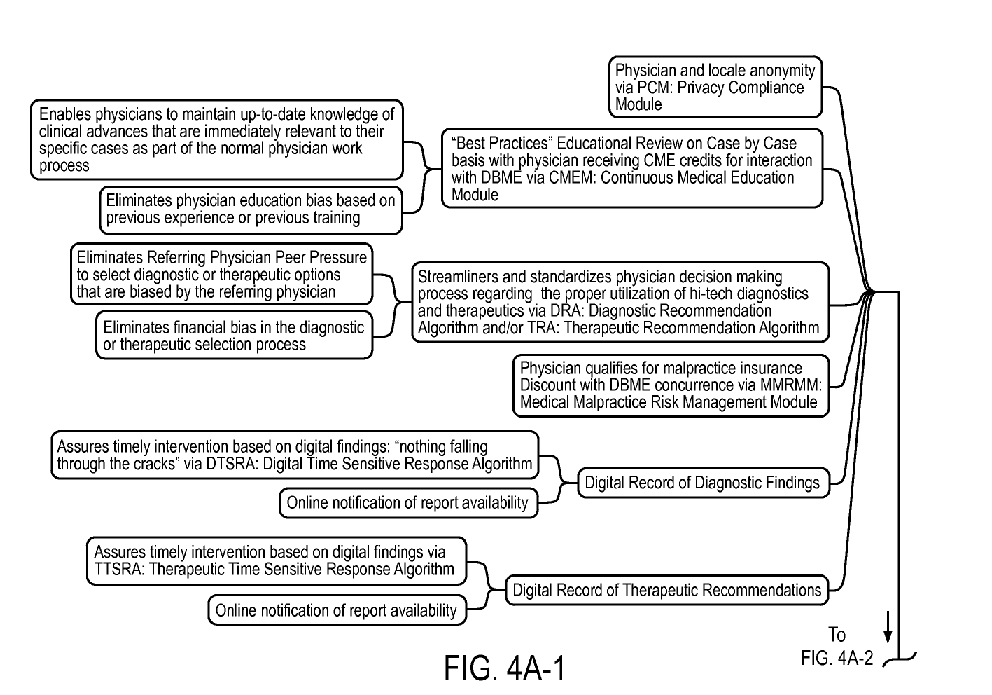

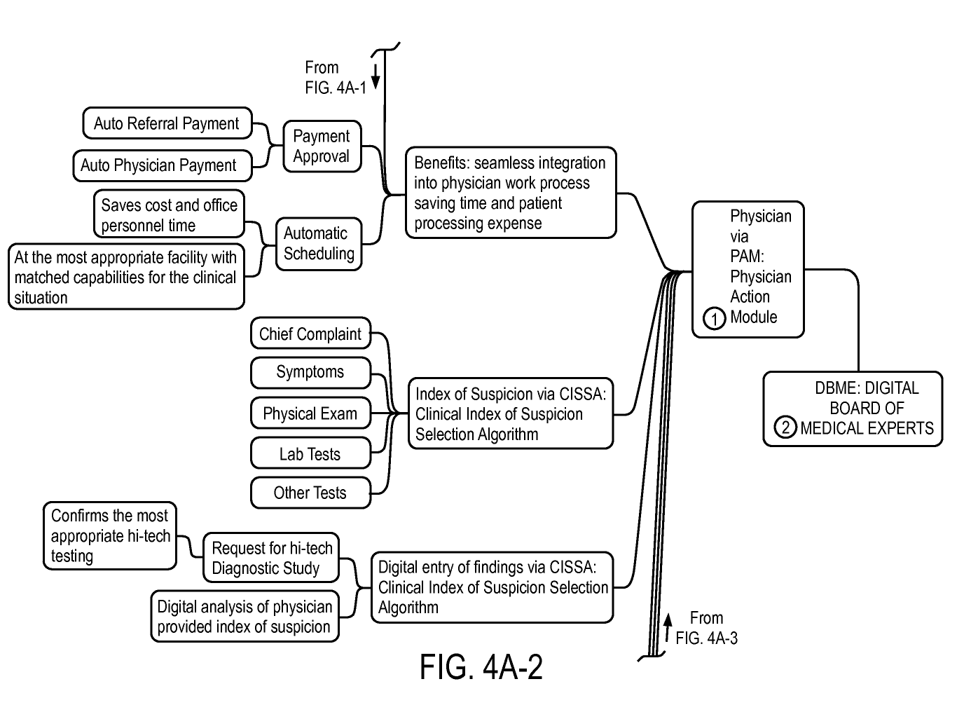

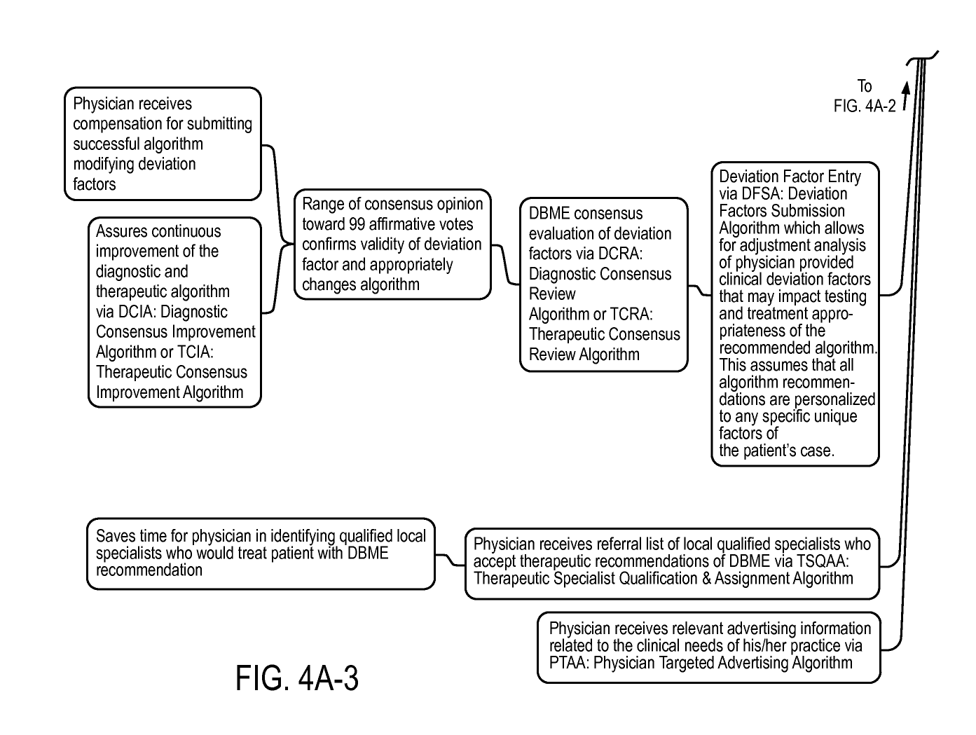

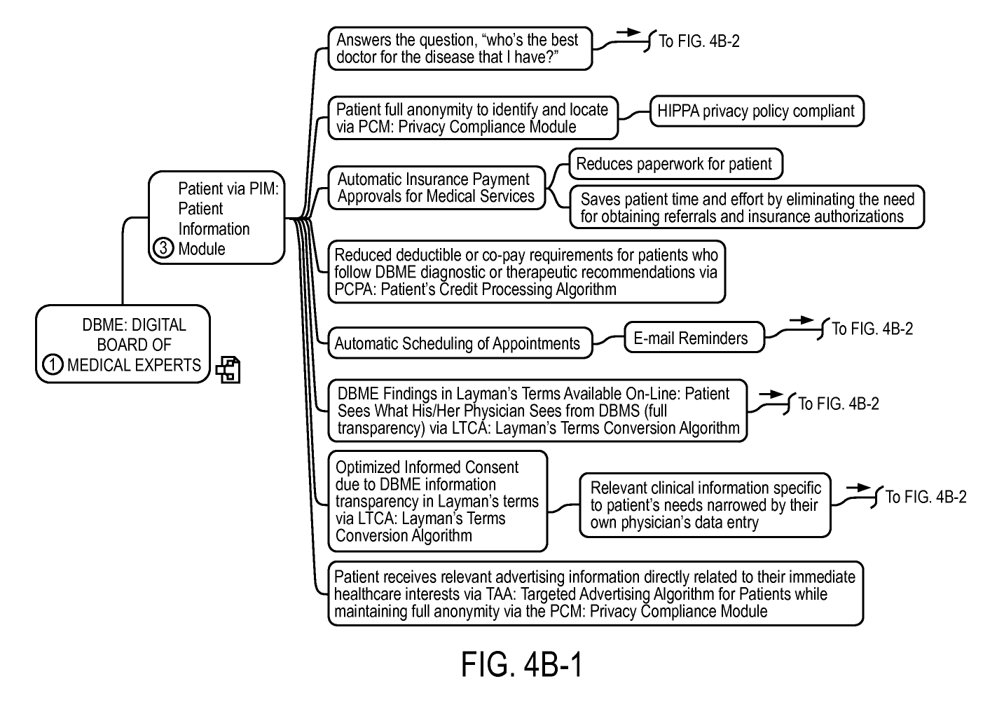

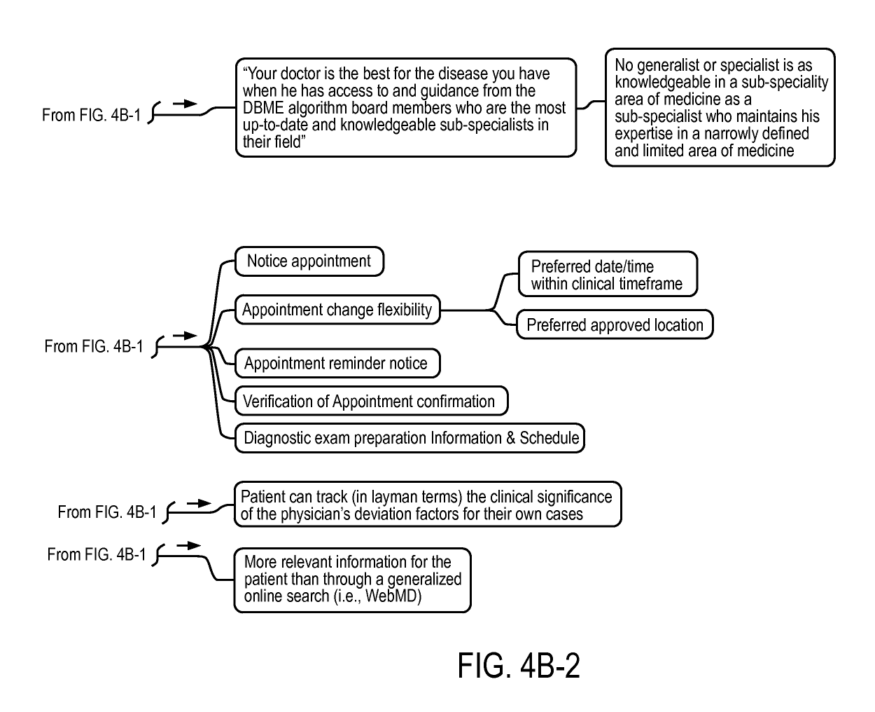

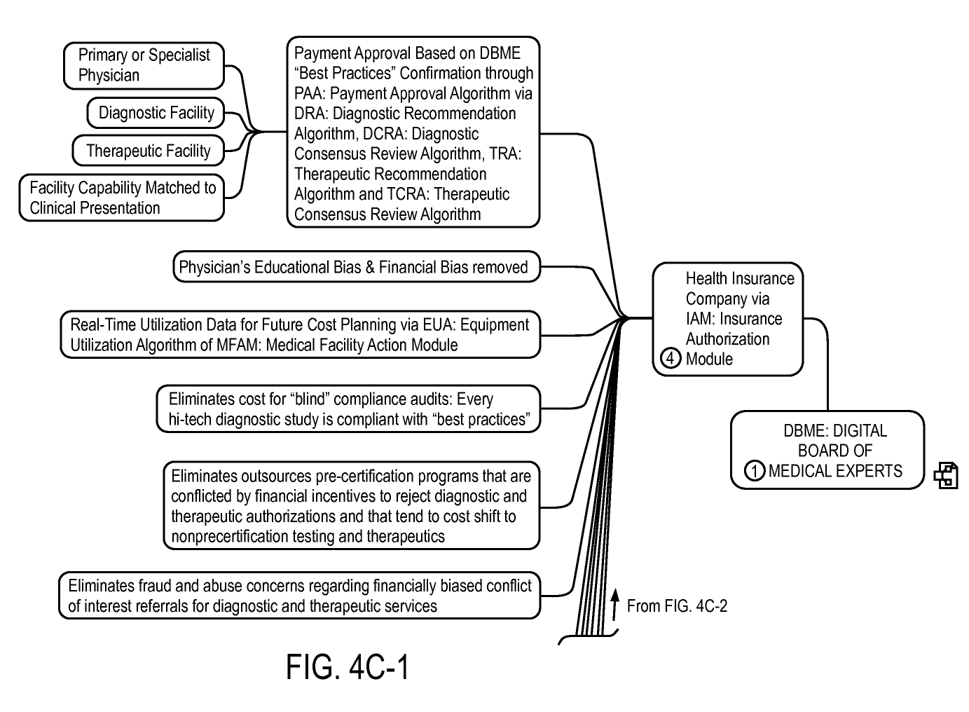

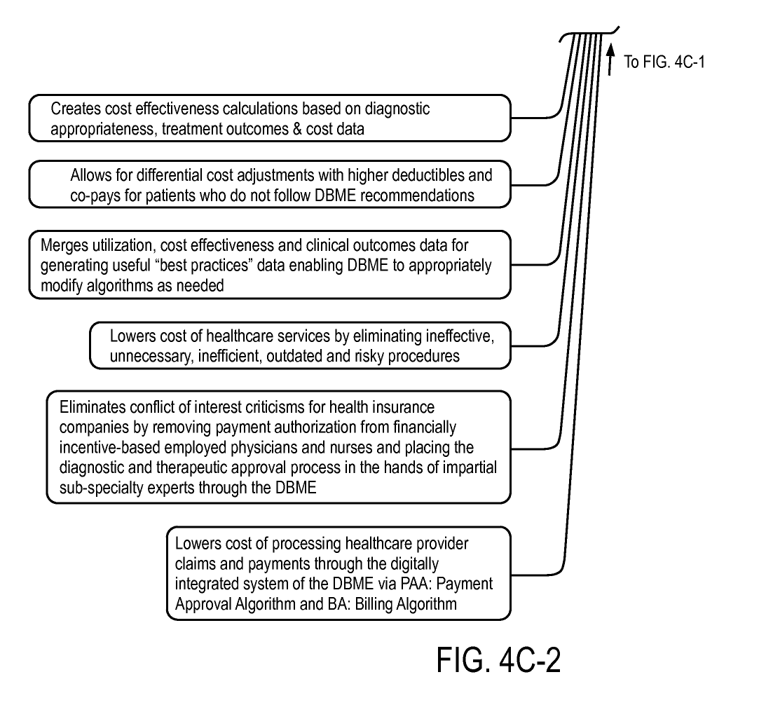

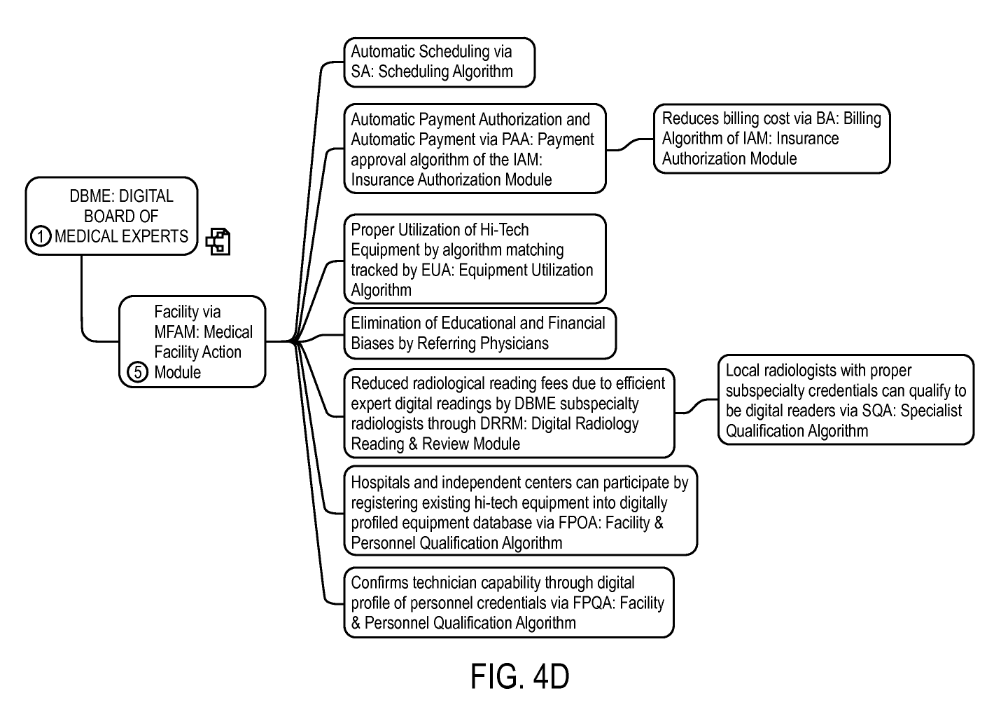

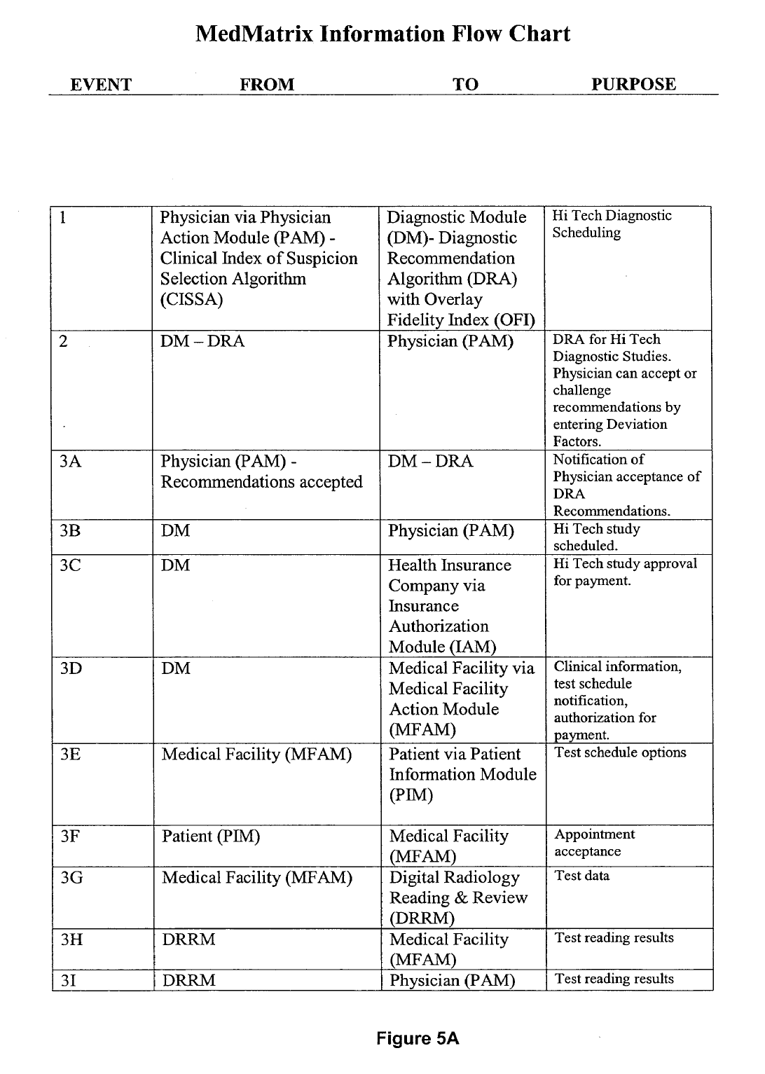

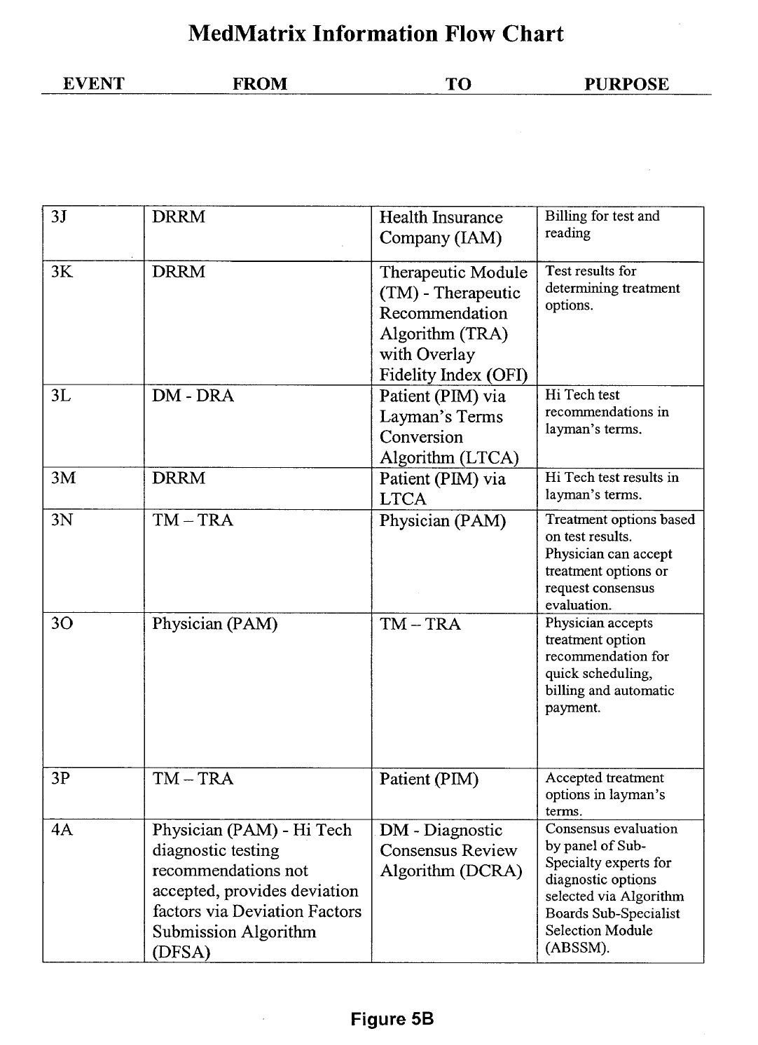

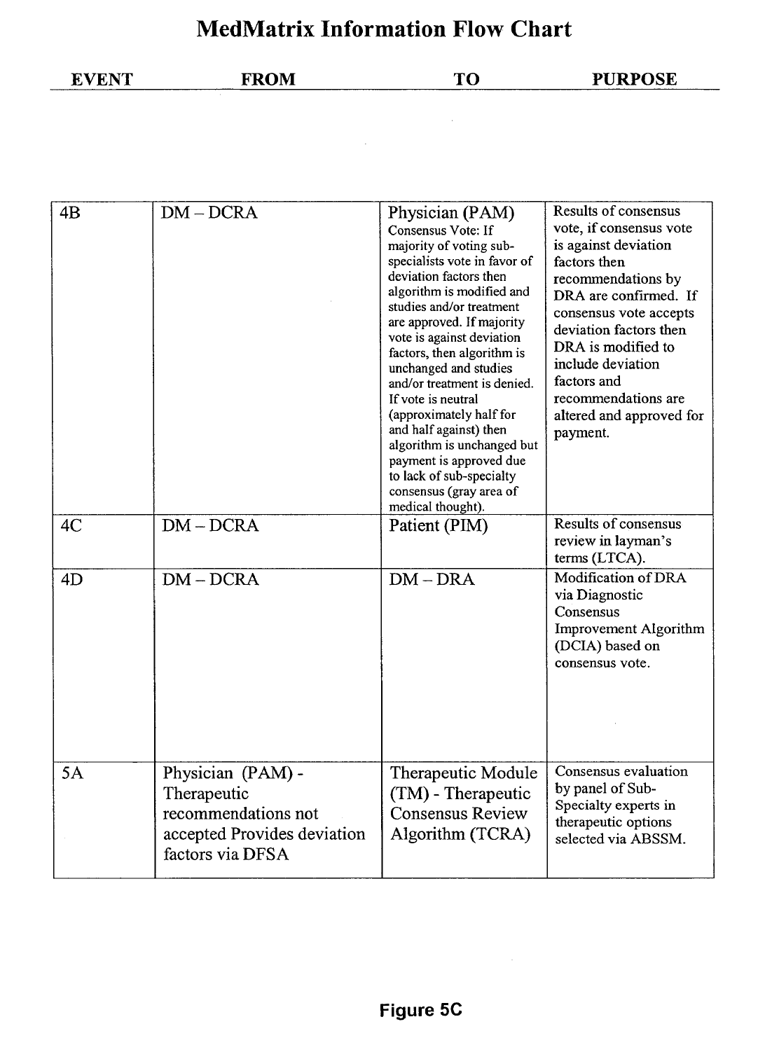

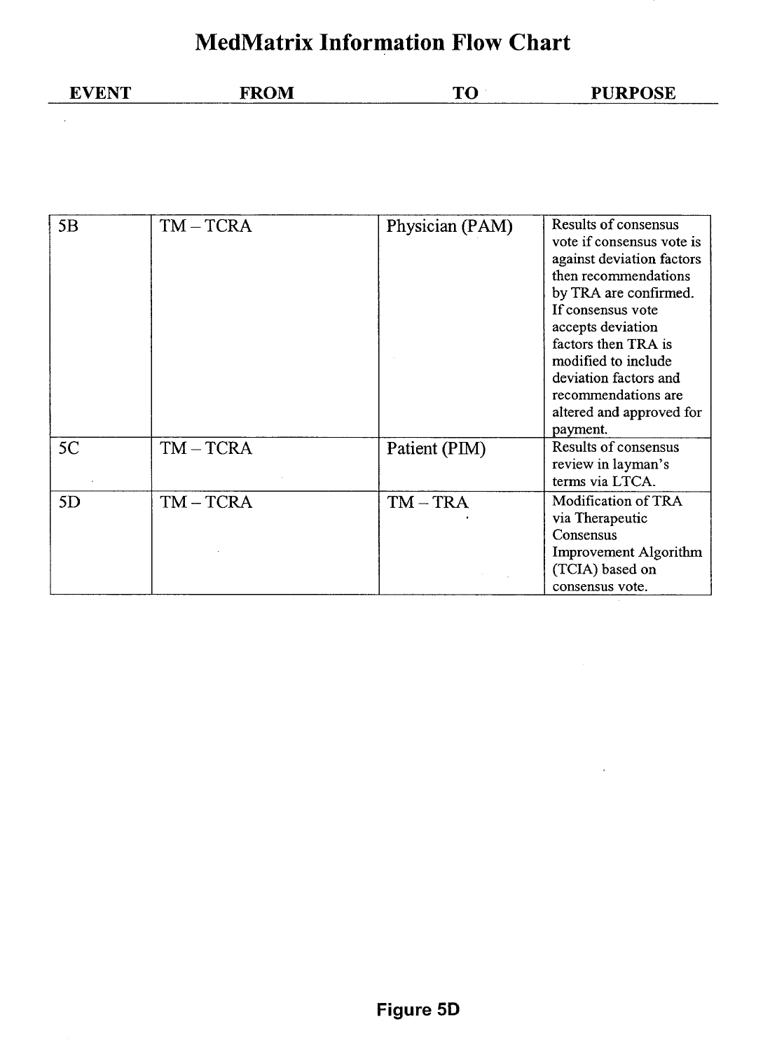

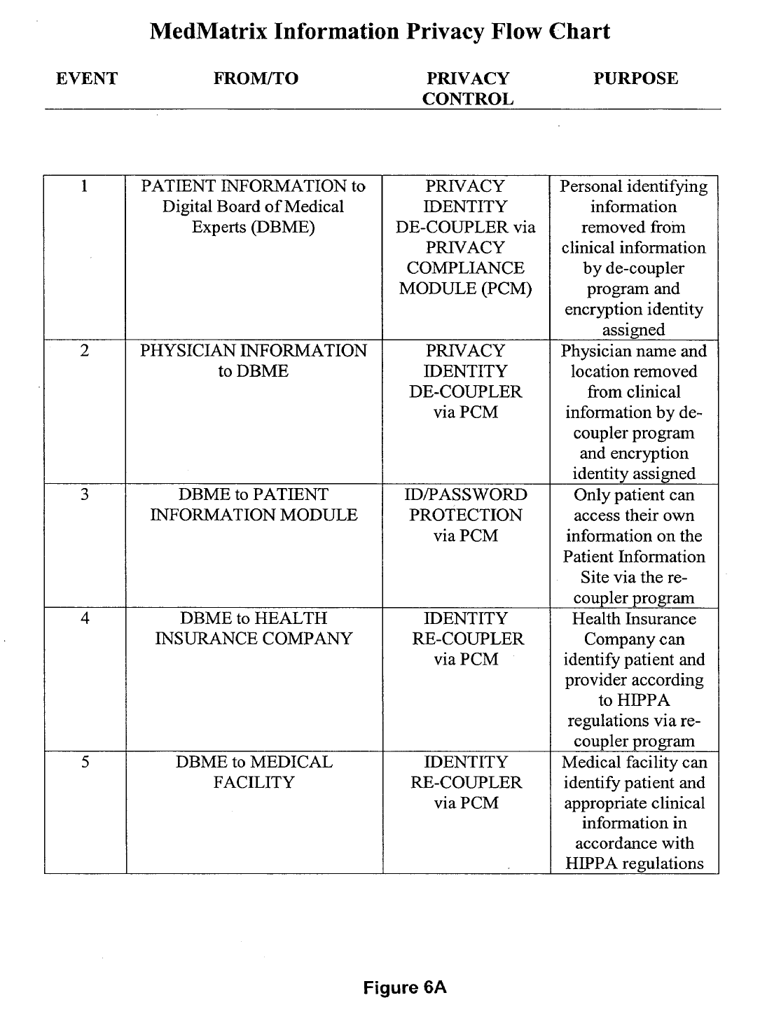

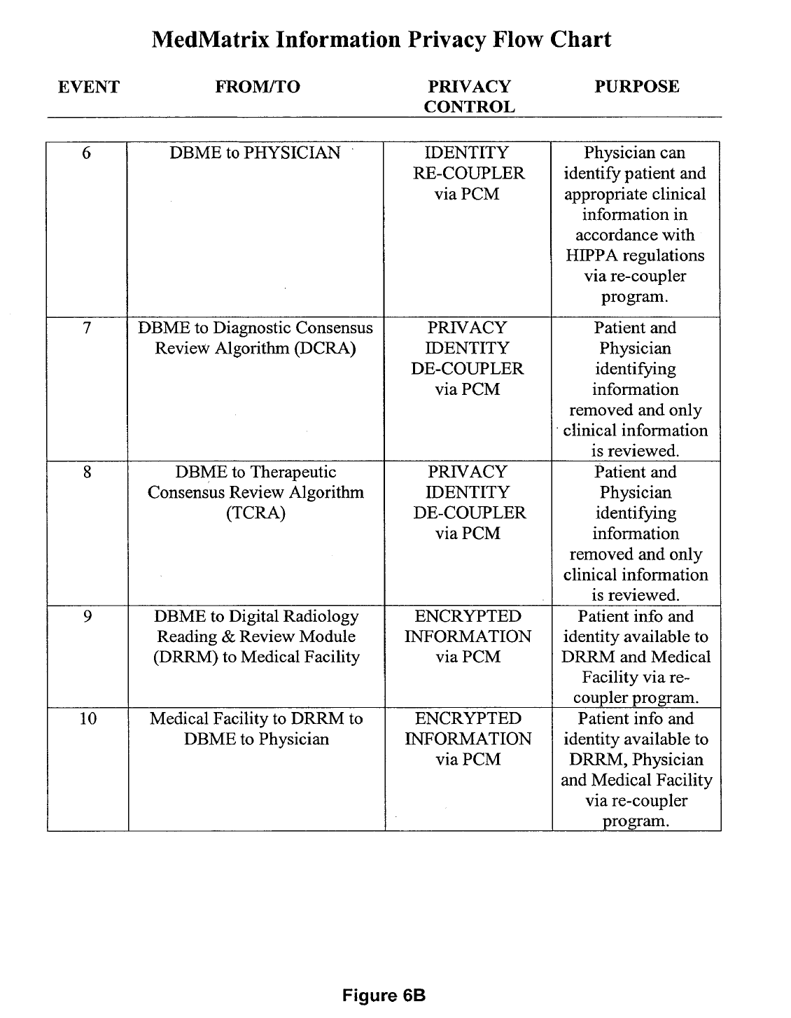

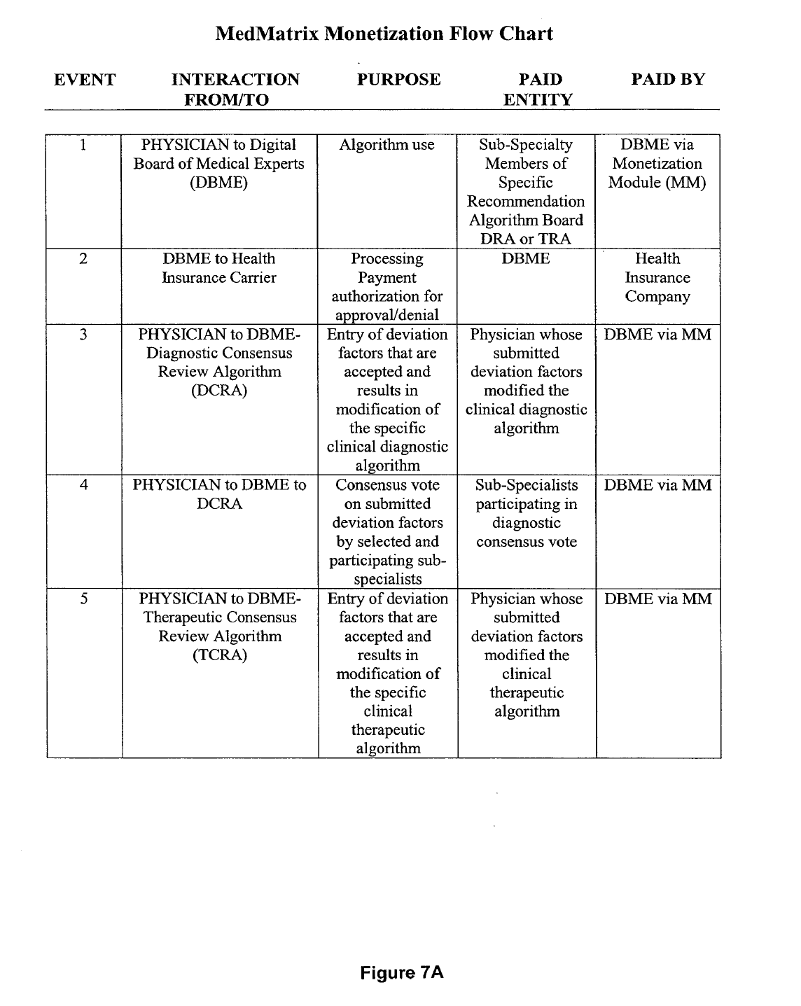

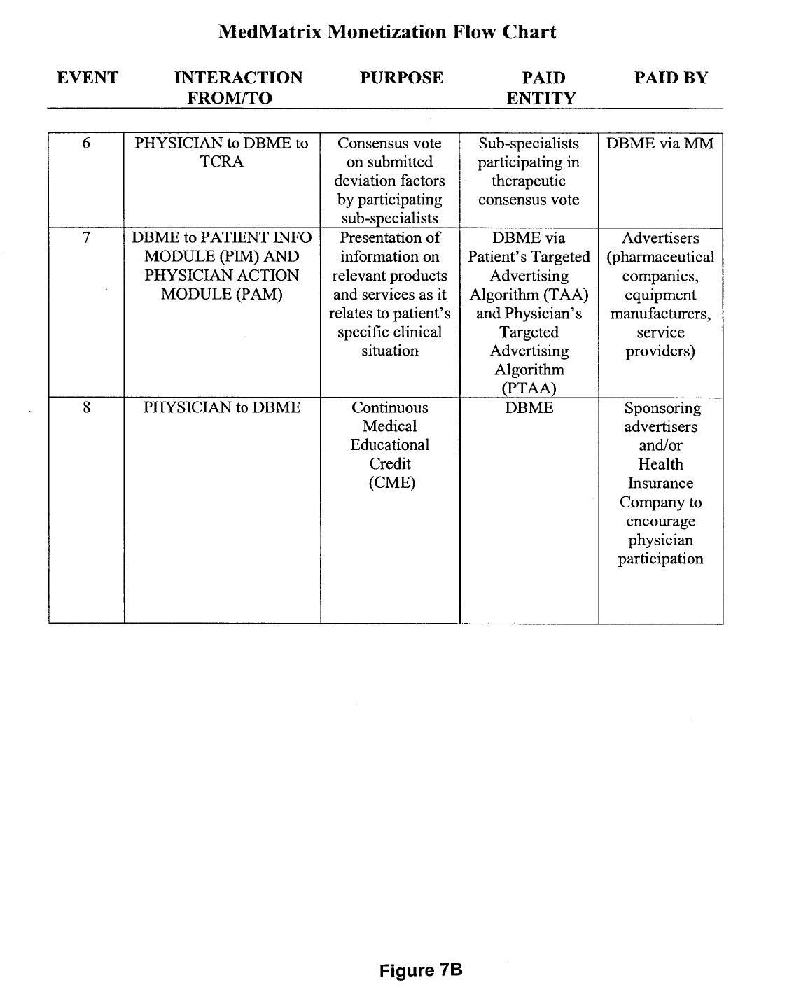

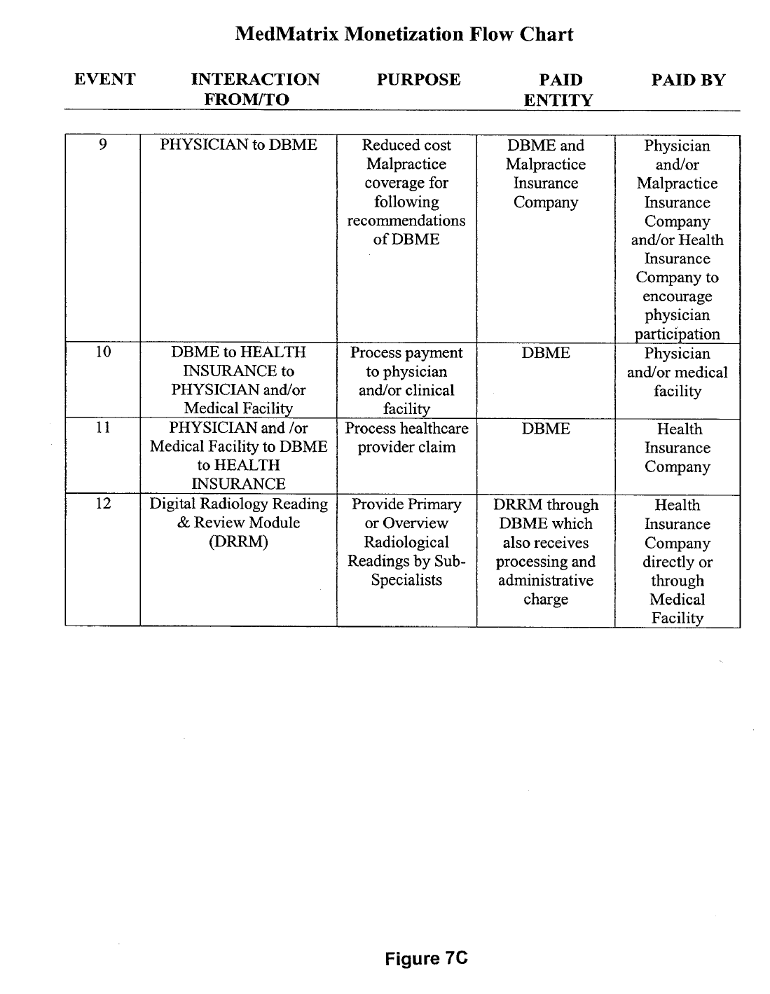

Embodiments of the present invention relate to a computerized method of providing information to any one of a plurality of patients for use in a medical diagnostic or treatment advice system on a computer network, the method comprising; accessing a portion of the patient medical history during an evaluation process, wherein each patient is associated with at least one file containing medical information unique to the medical condition of the patient, selectively executing at least one medical algorithm comprising accessing a database populated with data generated through a digital board of medical experts (DBME); and providing the medical advice to the selected patient; wherein the medical advice comprises an unbiased recommendation for a diagnostic test. The method of the invention is advantageous in that only a small portion of the patient's medical history is accessed in order to generate the recommendation. The accessed information is preferably related to an area at the cross-roads of a medical decision making process in medical practice. In particular, the information is related to high tech diagnostics relating to, without limitation CT, MRI, PET/CT and SPECT. One aspect of the invention relates to a digital analysis of physician provided clinical index of suspicion. In one embodiment, the clinical index of suspicion is based on an evaluation comprising symptom analysis, physical exam, lab tests and/or other tests results. FIGS. 1-7 illustrate embodiments of the invention. FIG. 1A shows modules such as the Physician Action Module and the Diagnostic Module. FIG. 1B shows modules including the Privacy Compliance Module. FIG. 2A illustrates how information flows to a nationwide sub-specialty board. FIG. 2B further illustrates flow of information to sub-specialty components. FIG. 3 illustrates flow of information to Digital Board of Medical Experts. FIG. 4A-1 illustrates streamlining of physician decisions. FIG. 4A-2 shows incorporation of Index of Suspicion via CISSA. FIG. 4A-3 shows Deviation Factor Entry via DFSA. FIG. 4B-1 shows information flow through a Patient Information Module. FIG. 4B-2 illustrates patient tracking of clinical significance of the physician's deviation factor. FIG. 4C-1 illustrates information flow to Insurance Authorization Module. FIG. 4C-2 shows incorporation of a Payment Approval Algorithm. FIG. 4D illustrates flow of information through a Medical Facility Action Module. FIG. 5A shows a sequence of events including test schedule notification and authorization for payment. FIG. 5B illustrates sequence of events including physician acceptance of treatment option. FIG. 5C illustrates sequence of events including consensus evaluations by panel of sub-specialty experts. FIG. 5D illustrates events including improvement of algorithm based on consensus vote. FIG. 6A illustrates sequence of events including removing personal indentifying information. FIG. 6B illustrates events including removing patient and physician identifying information. FIG. 7A illustrates algorithm use. FIG. 7B illustrates events including consensus vote on submitted deviation factors. FIG. 7C illustrates events such as processing payment to physician.

The invention provides a computerized method of providing information to any one of a plurality of patients for use in a medical diagnostic or treatment advice system on a computer network, the method comprising the following steps

A the physician provides a clinical index of suspicion via the clinical index of suspicion selection algorithm (CISSA) to the DBME—for obtaining a recommendation from the diagnostic recommendations algorithm (DRA) for hi tech diagnostic scheduling.

B the DBME processes the clinical index of suspicion and provides a recommendation through the diagnostic recommendation algorithm (DRA) to the Physician for hi tech Diagnostic Studies, whereby the Physician can accept or challenge recommendations by entering Deviation Factors.

The invention provides a method further comprising:

C Physician notifies the DBME of Physician acceptance of DRA Recommendations; and

D DBME notifies the Physician of hi tech study scheduled

The invention provides a method further comprising

E DBME notifies a Health Insurance Company of hi tech study approval for payment.

The invention also provides a method further comprising

F DBME identifies a Medical Facility and issuing clinical information, test schedule notification, and authorization for payment

The invention also provides a method further comprising:

G Medical Facility provides Patient Information Module (PIM) Test schedule options; and

H Patient provides via PIM to Medical Facility Appointment acceptance.

The invention provides a method further comprising:

I DBME provides test results from Medical Facility to Digital Radiology Reading and Review Module (DRRM);

J DRRM provides Test reading results to DBME; and

K DBME forwards to Physician the Test reading results

The invention also provides a method of further comprising:

L DBME runs therapeutic recommendation algorithm (TRA) and processes Test results for determining treatment options.

The invention also provides a method of further comprising:

M DBME provides on Patient Information Module hi tech test recommendations in layman's terms;

N DBME—provides on Patient Information Module hi tech test results in layman's terms; and

O DBME—Treatment options based on test results. Physician can accept treatment options or request consensus evaluation of deviation factors, whereby Physician accepts treatment option recommendation for quick scheduling, billing and automatic payment; and

P DBME provides on Patient Information Module Accepted treatment options in layman's terms.

The invention provides a method further comprising:

C′ Physician notifies DBME of hi tech diagnostic testing recommendations not accepted)

D′ Physician provides deviation factors DBME—

E′ DBME submits deviation factors to diagnostic consensus review algorithm (DCRA) for vote by selected subspecialty experts;

F′ Consensus evaluation conducted by panel of sub specialty experts for diagnostic options.

The invention also provides a method further comprising:

G′ DBME conducts Consensus Vote:
(i) If majority of voting sub-specialists vote in favor of deviation factors then algorithm is modified and studies and/or treatment are approved;
   (ii) If majority vote is against deviation factors, then algorithm is unchanged and studies and/or treatment is denied;
(iii) If vote is neutral (approximately half for and half against) then algorithm is unchanged but payment is approved due to lack of sub-specialty consensus (gray area of medical thought)

The invention also provides a method further comprising:

H′ DBME provides Results of consensus vote:

(i) if consensus vote is against deviation factors then recommendations by DRA are confirmed;

(ii) If consensus vote accepts deviation factors then DRA is modified via diagnostic consensus improvement algorithm (DCIA) which is a self-improving program to include accepted deviation factors into the recommendation algorithms which are self-adjusting.

The invention also provides a method further comprising:

I′ DBME provides on Patient Information Module Results of consensus review in layman's terms.

The invention also provides a method further comprising:

J′ DBME—modifies DRA via DCRA and DCIA based on consensus vote.

The invention also provides a method further comprising:

K′ Physician notifies DBME of therapeutic recommendations not accepted and Provides deviation factors;

L′ DBME submits deviation factors to therapeutic consensus review algorithm (TCR);

M′ Consensus evaluation is conducted by panel of sub specialty experts in therapeutic options selected by ABSSM.

The invention also provides a method further comprising:

N′ DBME provides to Physician Results of consensus vote:
(i) if consensus vote is against deviation factors then recommendations by TRA are confirmed;
(ii) If consensus vote accepts deviation factors then TRA is modified to include deviation factors and recommendation are altered and approved for payment.

The invention also provides a method further comprising:

O′ DBME provides on Patient Information Module results of consensus review in layman's terms.

The invention also provides a method further comprising:

P′ DBME implements Modification of TRA via TCRA and TCIA based on consensus vote.

The invention also provides a method of Claim further comprising:

Q′ DBME presents to patient information on relevant products and services as it relates to patient's specific clinical situation including information through DBME/Search Engine

Advertisers (pharmaceutical companies, equipment manufacturers, service providers).

The invention also provides a method further comprising

R′ DBME provides to physician Continuous Medical Educational (CME) Credit through CMEM and/or sponsoring advertisers and/or Health Insurance Company to encourage physician participation

The invention also provides a method further comprising:

DBME provides to physician reduced cost Malpractice coverage or credit against medical malpractice premiums for following recommendations of DBME.

### EXAMPLE

This example illustrates, without limitation, implementation of selected embodiments of the invention in connection with a patient having lung cancer.

1) 61 year white male patient (p) presents to family physician (Fp) with complaint of coughing up bloody sputum. Fp does physical exam, obtains chest x-ray in office, draws blood samples and sends sputum sample for cytology. Results of chest x-ray shows 2.5 cm mass in the left middle lobe of the lung. Cytology is positive for adenocarcinoma. Blood tests are negative.

2) Fp enters clinical index of suspicion via Clinical Index of Suspicion Selection Algorithm (CISSA) of the Physician Action Module (PAM) in order to obtain work-up to evaluate metastases with CT chest, abdomen, brain, and obtain a referral for surgery.

3) CISSA communicates data to Diagnostic Module (DM) of DBME for confirmation of clinical plan. Diagnostic Recommendation Algorithm (DRA) of the DM matches CISSA data via an Overlay Fidelity Index (OFI) of the appropriate DRA for the identified disease process. All patient and physician identifying information is encrypted by the Privacy Compliance Module (PCM) to assure total privacy.

4) The matched DRA recommends whole body PET/CT scan which is a metabolic study capable of identifying the presence of cancer that has spread from its primary site (the lung) to other sites by not only changes in size but also in sites where is there is no differences in size by the differential uptake of marker metabolites. PET/CT in this case is more accurate for properly staging the disease but also is 50% less expensive than the typical multiple CAT scans originally requested. The DRA also requests that cancer cells from the sputum be sent for pharmaco-genetic profile of the cancer to determine which chemotherapeutic agents would be most effective against this patient's specific cancer cells and also cells from the patient (inner cheek swab) be sent for pharmaco-genetic profiling in order to determine which chemotherapeutic agents would have the least negative effects for the patient. The Fp receives, along with the diagnostic recommendations, detailed up-to-date medical literature review and references supporting the clinical logic and practicality of the specific recommendations as they specifically relate to the case and based on the known information about the patient at this point in the case evaluation. The DAR also communicates the information to the Diagnostic Time Sensitive Response Algorithm (DTSRA) because there is a life-threatening disease process being evaluated and patient's evaluation must proceed in a timely manner.

The DTSRA will remind all parties involved of the optimal time-frame for completion of the clinical tasks necessary to properly manage the patient's disease. Identical information is also sent to the patient through the Patient Information Module (PIM) but the information is translated into layman's terms by the Layman's Terms Conversion Algorithm (LTCA).

5) The Fp receives the recommendations of the DRA, whereby the Fp acknowledges them and accepts them as a course of action for the patient. Then the Fp also receives a continuous medical education credit for acknowledging the medical literature review for this specific case through the Continuous Medical Education Module (CMEM) and the Fp also receives automatic medical malpractice coverage for this case at this point in time as a credit against his overall medical malpractice premium cost through the Medical Malpractice Risk Management Module (MMRMM). The Fp also has his/her bill seamlessly generated and automatically submitted for payment through the Monetization Module (MM) and the Billing Algorithm (BA) and the Payment Approval Algorithm (PAA) of the Insurance Authorization Module (IAM) which communicates with the patient's health insurance company. The Fp bills will be automatically generated and approved for payment for the all clinical services provided by the Fp throughout the entire management process of the patient as the Fp communicates with the DBME. The patient is scheduled for PET/CT studies at the nearest qualified medical facility through the Facility & Personnel Qualification Algorithm (FPQA) of the Medical Facility Action Module (MFAM). An appointment is generated by the Scheduling Algorithm (SA) of the MFAM and communicated to the patient through the PIM for acceptance. The patient also receives information on the co-pay and/or deductible credits available to the patient for acceptance of the proposed studies as well as a cost comparison of the co-pay and/or deductible expenses for the proposed studies through the Patient's Credit Processing and Cost Comparison Algorithm (PCPA) of the PIM. The medical facility receives payment authorization from the PAA of the IAM and will also receive automatic billing from the MM and the BA of the IAM seamlessly once the studies are completed satisfactorily.

6) After the patient accepts the appointment and undergoes the PET/CT studies, the study data is communicated to the Digital Radiology Reading and Review Module (DRRM) to verify the quality of the test data obtained and the radiological reading on which subsequent key clinical decisions will be made. After the PET/CT study data has been reviewed through the Test Quality Assessment Algorithm (TQAA) and the Test Reading Quality Assessment Algorithm (TRQAA), the information is communicated to the Therapeutic Module (TM) and matched by the OFI to the appropriate Therapeutic Recommendation Algorithm (TRA) and the results of the PET/CT studies and matched TRA are communicated to the Fp through the PAM along with input from the Therapeutic Time Sensitive Response Algorithm (TTSRA) to remind all parties of the time critical decisions that made to maximize a favorable outcome.

7) Results of the PET/CT studies indicate no distant metastases but locally advanced disease is identified in the lung which has spread to both the hilar lymph nodes and the mediastinal lymph nodes. The TRA recommendation is combination chemotherapy with radiation therapy. Based on the pharmaco-genetic studies of both the cancer cells and the patient's own normal cells, it is determined a specific two drug combination will achieve the best response whereby the cancer cells will be most susceptible to the drug combination but the patient will be most tolerant to this personalized drug combination in terms of less side effects. Furthermore, to limit the side effects of one of the drugs and based on the pharmaco-genetic profile of the patient, it is recommended that the drug in question is administered in the evening rather than in the morning. The other drug is to be administered through a low-dose continuous infusion method which will minimize the side-effects and maximize the synergistic effects of the combined radiation therapy. The radiation therapy is recommended to be performed on a state of the art image-guided tomotherapy unit which combines real-time targeting through the built-in accuracy of a CAT scan within the radiation therapy unit that also utilizes multiple narrow beams generated from numerous angles to geometrically converge over the tumor mass and the adjacent lymph nodes thereby insuring the maximum cancer shrinkage with the least amount of side-effects so that the therapy will not be a debilitating experience. The Fp receives, along with the therapeutic recommendations, detailed up-to-date medical literature review and references supporting the clinical logic and practicality of the specific recommendations as they specifically relate to the case and based on the known information about the patient at this point in the case evaluation. The TAR also communicates the information to the Therapeutic Time Sensitive Response Algorithm (TTSRA) because there is a life-threatening disease process and the patient's treatment must proceed in a timely manner. The TTSRA will remind all parties involved of the optimal time-frame for completion of the clinical tasks necessary to properly manage the patient's disease. Identical information is also sent to the patient through the Patient Information Module (PIM) but the information is translated into layman's terms by the Layman's Terms Conversion Algorithm (LTCA) for maximum understanding and facilitation of optimal informed consent by the patient.

8) The Fp is asked to acknowledge and approve the treatment recommendations. However, after reviewing the test results and recommendation, Fp does not agree with the recommendations for chemotherapy and radiation therapy but prefers that the patient undergo surgery first to give the patient a better chance for a cure. Therefore, the Fp enters deviation factors through the Deviation Factors Submission Algorithm (DFSA) of the PAM which the Fp believes will alter the recommendation of the TRA. The DFSA information is sent to the Therapeutic Consensus Review Algorithm (TCRA) for a consensus vote of sub-specialty experts in the field of clinical disease under evaluation.

The experts are selected through an OFI match of the ABSSM. The consensus vote results in a vote of 49 to 0 in favor of the TRA recommendation. The overwhelming medical evidence suggests that the overall survival time is reduced five fold in pursuing surgery first in the case in question because of the pre-existence of spread of the cancer to the lymph nodes which will not be contained or encompassed by the surgery. Furthermore, surgery will result in complications and side effects that will delay definitive treatment and allow the cancer cells to grow and spread while the patient is recovering from surgery. Furthermore, vital nutritional and immunological reserves of the patient will be depleted during the post-operative convalescence period placing the patient at higher risk for complications and lower tolerance for subsequent chemotherapy and radiation therapy which will still be required after surgery. The results of the TCRA are communicated to both the Fp through PAM and the patient through the LTCA of the PIM. The physician and patient agree to proceed with the chemotherapy and radiation therapy as recommended. The Fp receives a list of qualified medical oncologist and radiation oncologists familiar and expert in delivery the course of therapy recommended by the TRA through the Therapeutic Specialist Qualification and Assignment Algorithm.

9) Once the medical oncologist and radiation oncologist are selected from the TSQAA by the Fp and agreed to by the patient, the patient is scheduled for an appointment through the physicians' offices through the PAM or at their medical facilities through the MFAM. Seamlessly, payment approval for the treatments is processed by the PAA of the IAM and the PCPA of the PIM reducing processing cost and time for the physician, patient and medical facility.

10) The process of expert sub-specialty oversight and confirmation is continued by the medical oncologist and radiation oncologist as they enter their more specialized information into the DBME for further guidance from nationally recognized sub-specialty experts and for seamless processing of payments approvals, billing, CME credits, medical malpractice premium credits and patient management data support.

11) The elimination of unnecessary surgery in this case resulted not only in a better overall survival and improvement in quality of life during therapy but also saved over $60,000 in unnecessary costs that would not have benefited the patient. Over 80% of so-called curative lung cancer surgeries result in an up-staging of the disease (disease identified to be further advanced than originally believed to be before surgery as a result of utilizing only CAT scans and not the more definitive PET/CT scans). This results in billions of dollars of wasted healthcare expenditures annually for just this one type of disease presentation

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A computerized method of managing medical care through communication between a Digital Board of Medical Experts (DBME), and one or more of a physician; an insurance carrier; one or more medical facilities; and a patient and providing a diagnostic and/or therapeutic recommendation, the method comprising the following steps
i) providing a DBME comprising modules and algorithms for processing medical data and providing diagnostic and/or therapeutic recommendations;
ii) providing a Physician Action Module (PAM) whereby a physician provides a clinical index of suspicion (CIS) through a clinical index of suspicion selection algorithm (CISSA) to the DBME—for obtaining a recommendation for hi tech diagnostic scheduling and/or treatment; and
iii) processing through the DBME the clinical index of suspicion and providing a recommendation through a diagnostic recommendation algorithm (DRA) for hi tech diagnostic studies and/or through a therapeutic recommendation algorithm (TRA) for treatment recommendations.

2. The method of claim 1, wherein the DBME comprises one or more of a diagnostic module (DM), a therapeutic module (TM), a digital radiological reading and review module (DRRM), a patient information module (PIM), a physician action module (PAM), a health insurance authorization module (IAM), a medical facility action module (MFAM), a continuous medical education module (CMEM), a monetization module (MM), a medical malpractice risk management module (MMRMM), a privacy compliance module (PCM), and a algorithm boards sub-specialist selection module (ABSSM).

3. The method of claim 2, wherein the PAM comprises a clinical index of suspicion selection algorithm (CISSA), a deviation factors submission algorithm (DFSA), a therapeutic specialist qualification and assignment algorithm (TSQAA), and a physician targeted advertising algorithm (PTAA).

4. The method of claim 3, further comprising selection by the physician of a CIS from a CIS menu processed through the CISSA.

5. The method of claim 2, wherein the PIM comprises a layman's terms conversion algorithm (LTCA), a targeted advertising algorithm for patients (TAA), and a patient's credit processing and cost comparison algorithm (PCPA).

6. The method of claim 2, wherein the DM comprises one or more of a diagnostic recommendation algorithm (DRA), diagnostic consensus review algorithm (DCRA), a diagnostic consensus improvement algorithm (DCIA), and a diagnostic time sensitive response algorithm (DTSRA).

7. The method of claim 2, wherein the TM comprises one or more of a therapeutic recommendation algorithm (TRA), a therapeutic consensus review algorithm (TCRA), a therapeutic consensus improvement algorithm (TCIA), a prevention recommendation algorithm (PRA), and a therapeutic time sensitive response algorithm (TTSRA).

8. The method of claim 2, wherein the MFAM comprises one or more of a facility and personnel qualification algorithm (FPQA), a specialist qualification algorithm(SQA), a scheduling algorithm (SA), and an equipment utilization algorithm (EUA).

9. The method of claim 2, wherein the DRRM comprises one or more of a test quality assessment algorithm (TQAA), a test reading quality assessment algorithm (TRQAA), a payment proration algorithm (PPA); a performance tracking algorithm (PTA); and a reading specialist qualification and assignment algorithm (RSQAA).

10. The method of claim 2, wherein the IAM comprises a payment approval algorithm (PAA) and/or a billing algorithm (BA).

11. The method of claim 1, wherein the CIS comprises a presenting complaint.

12. The method of claim 8, wherein the presenting complaint comprises a chief presenting complaint.

13. The method of claim 8 wherein the presenting complaint comprises a secondary presenting complaint.

14. The method of claim 8, wherein the CIS comprises a physician adjusted chief complaint.

15. The method of claim 1, wherein the CIS comprises symptomatology derived factors.

16. The method of claim 12, wherein the symptomatology derived factors are based on patient complaints.

17. The method of claim 1, wherein the CIS comprises past medical history factors.

18. The method of claim 14, wherein the past medical history comprises prior treatments.

19. The method of claim 15, wherein the prior treatment comprises one or more of non-surgical treatment and surgical treatment.

20. The method of claim 16, wherein the non-surgical treatment comprises one or more of non-invasive procedure and medications.

21. The method of claim 1, wherein the CIS comprises physical examination findings.

22. The method of claim 1, wherein the CIS comprises results of laboratory tests.

23. The method of claim 19, wherein the laboratory tests comprises testing body fluids.

24. The method of claim 20, wherein the body fluid is selected from blood, urine, spinal fluid, sputum, or other types of body fluids.

25. The method of claim 1, wherein the CIS comprises the results of imaging test.

26. The method of claim 1, wherein the CIS comprises the results of cellular or tissue pathology findings.

27. The method of claim 23, wherein the imaging tests comprise X-ray, radionuclear and ultrasound.

28. The method of claim 1, wherein the CIS comprises asymptomatic profile.

29. The method of claim 1, wherein the CIS comprises a genetic profile.

30. The method of claim 1, wherein the CIS comprises environmental profile.

31. The method of claim 1, wherein the CIS comprises a behavioral profile.

32. The method of claim 28, wherein the behavioral profile comprises alcohol drinking habits, nicotine intake habits; narcotic or other addictive substance use.

33. The method of claim 1, wherein the CIS comprises family history profile.

34. The method of claim 1, wherein the CIS comprises a proposed High tech diagnostic test.

35. The method of claim 1, wherein the DRA comprises determining an overlay fidelity index (OFI) between the CIS provided by the physician and DBME defined factors.

36. The method of claim 32, wherein the overlay fidelity index required by the DRA is determined based on the type of test, the invasiveness of the test, risk to the patient associated with the test and cost of the test.

37. The method of claim 32, wherein the overlay fidelity index required by the DRA is adjusted based on cost effectiveness data.

38. The method of claim 34, wherein the overlay fidelity index is adjusted based on predetermined percentage of false positive and/or false negative outcomes.

39. The method of claim 34, wherein the cost effectiveness data is based on CIS appropriateness, diagnostic outcomes, and cost data.

40. The method of claim 32, wherein the DRA provides a recommendation based on a CIS having an overlay fidelity index of 50% or greater.

41. The method of claim 32, wherein the DRA comprises requests for additional CIS factors based on a threshold overlay fidelity index.

42. The method of claim 32, wherein factors in the CIS comprises weighting factors.

43. The method of claim 1, wherein the high tech diagnostic test comprises an invasive procedure.

44. The method of claim 43, wherein the invasive procedure is selected from a colonoscopy, cystoscopy, arteriography, cholecystography, endoscopy, laparoscopy, and mediastinoscopy or other methods requiring visualization of internal organs.

45. The method of claim 1, wherein the high tech diagnostic test comprises a non-invasive procedure.

46. The method of claim 45, wherein the non-invasive procedure is selected from CAT scans, radionuclear scans, PET scans, MRI, and ultrasound imaging.

47. The method of claim 1, wherein the DM and TM of the DBME are formed by recommendation algorithms (DRA, TRA, PRA) developed by experts who are identified and chosen through the ABSSM.

48. The method of claim 47, wherein the experts are organized in DCRA, DRA, TRA, TCRA and RSQAA boards.

49. The method of claim 47, wherein the algorithms are digitally linked for immediate access for diagnostic and therapeutic evaluations and recommendations.

50. The method of claim 47, wherein the DBME comprises algorithms for clinical presentations that have high impact probability for better outcomes with lower costs.

51. The method of claim 47, wherein the DBME comprises algorithms supported by a board of experts grouped according to a set of subspecialties.

52. The method of claim 51, wherein the experts maintain the integrity and quality of the algorithm.

53. The method of claim 51, wherein the experts are selected through the ABSSM based on their publications, lectures, clinical experience, faculty affiliations, positions within medical specialty colleges and associations, government agencies, national and international bodies, foundations, clinics and hospitals.

54. The method of claim 47, wherein the DBME comprises algorithms supported by experts in radiology from MRI subspecialties including head and neck, neuro and brain, bone, chest, abdomen, pelvis, breast and cardiac imaging.

55. The method of claim 47, wherein the DBME comprises algorithms supported by experts in radiology from PET/CT subspecialties including brain, chest, abdomen, pelvis, and cardiac imaging.

56. The method of claim 47, wherein the DBME comprises algorithms supported by experts in radiology from CT subspecialties including brain, head and neck, chest, abdomen, pelvis, and cardiac imaging.

57. The method of claim 47, wherein the DBME comprises algorithms supported by experts in radiology and internal medicine from SPECT/Nuclear Medicine diagnostic and therapeutic subspecialties

58. The method of claim 47, wherein the DBME comprises algorithms supported by experts in cardiology, medical oncology, surgical oncology and radiation oncology.

59. The method of claim 58, wherein the DBME comprises algorithms supported by cardiologists having one or more subspecialties selected from pediatric cardiology, interventional cardiology, peripheral vascular, electrophysiology, and cardiac surgery.

60. The method of claim 58, wherein the DBME comprises algorithms supported by experts in medical oncology having one or more subspecialties selected from pediatric oncology, neuro-oncology, head and neck oncology, breast oncology, lung oncology, gastrointestinal oncology, gynecologic oncology and urologic oncology.

61. The method of claim 58, wherein the DBME comprises algorithms supported by experts in surgical oncology having one or more subspecialties selected from pediatric surgical oncology, neuro surgical oncology, head and neck surgical oncology, breast surgical oncology, thoracic surgical oncology, abdominal surgical oncology, colorectal surgical oncology, gynecologic surgical oncology, and urologic surgical oncology.

62. The method of claim 57, wherein the DBME comprises algorithms supported by experts in radiation oncology having one or more subspecialties selected from pediatric radiation oncology, neuro radiation oncology, head and neck radiation oncology, breast radiation oncology, lung radiation oncology, abdominal radiation oncology, colorectal radiation oncology, urologic radiation oncology, and gynecologic radiation oncology.

63. The method of claim 1, wherein the physician accepts the diagnostic recommendation of the DBME or challenges the recommendation and provides deviations factors for further consideration by the DBME.

64. The method of claim 1, wherein the DRA comprises a detailed description of the medical and scientific basis of the recommendation.

65. The method of claim 1, further comprising:
iv) providing notification from the physician to the DBME of the physician's acceptance of DRA recommendations.

66. The method of claim 65, further comprising processing CME credit award to the physician.

67. The method of claim 65, further comprising providing the physician with a certificate of credit for malpractice risk reduction.

68. The method of claim 65, further comprising:
v) providing notification from DBME to the physician of scheduled hi tech diagnostic study.

69. The method of claim 65, further comprising:
vi) providing notification from DBME to the Health Insurance Company of hi tech diagnostic study approval for payment.

70. The method of claim 65, further comprising:
vii) providing notification from DBME to the Medical Facility and
viii) issuing clinical information, test schedule notification, and authorization for payment

71. The method of claim 70, further comprising:
ix) providing through a Patient Information Module (PIM) Test schedule options available at the medical facility; and
x) providing via PIM to medical facility patient appointment acceptance.

72. The method of claim 71 further comprising:
xi) providing test results from the medical facility to Digital Radiology Reading and Review Module (DRRM).

73. The method of claim 71, further comprising:
xii) providing test results and a reading of the tests results from the medical facility to the Digital Radiology Reading and Review Module (DRRM).

74. The method of claim 72, further comprising:
xiii) checking through the DRRM the quality and comprehensiveness of the reading provided by the medical facility.

75. The method of claim 74, further comprising:
ixx) assessing through the DRRM the reading of the test results based on minimal subspecialty standards and accepting the reading or
xx) forwarding through the DRRM the test results to a subspecialty expert for further reading.

76. The method of claim 75, further comprising:
xxi) authorizing through the DRRM payment to the facility of a global fee if the reading provided by the facility is acceptable or
xxii) calculating and authorizing payment of a prorated fee if the reading was not accepted.

77. The method of claim 76, further comprising:
xxiii) processing through the TM the reading of the test results and determining whether a TRA is available for the findings, and if not forwarding the radiological readings to the physician without a therapeutic recommendation.

78. The method of claim 76, further comprising:
xxiv) processing through the TM the reading of the test results and determining whether a TRA is available for the findings, and if not forwarding to the physician a list of additional tests and/or results required to qualify for a TRA.

79. The method of claim 78, further comprising:
xxv) identifying through the TM a therapeutic algorithm for the findings and running the Therapeutic Recommendation Algorithm (TRA) to process the test results for determining treatment options.

80. The method of claim 79, further comprising:
xxvi) providing through the DBME treatment options to the physician based on test results.

81. The method of claim 80, wherein the physician can accept treatment options or request consensus evaluation based on submitted deviation factors.

82. The method of claim 81, further comprising upon physician acceptance of treatment option,
xxvii) recommending through the DBME to the physician a list of qualified facilities and specialists, and
xxviii) expediting billing and automatic payment.

83. The method of claim 81, further comprising:
xxix) providing through the Patient Information Module the hi tech test recommendations in layman's terms;
xxx) providing through the Patient Information Module the hi tech test results in layman's terms;
xxxi) providing through the Patient Information Module accepted treatment options in layman's terms; and/or
xxxii) providing through the Patient Information Module interactive tools to obtain informed consent from patient prior to acceptance of recommendations for diagnostic testing and or treatment.

84. The method of claim 1, further comprising:
xxxiii) providing notification from the physician to the DBME of hi tech diagnostic testing recommendations not accepted; and
xxxiv) obtaining from the physician deviation factors for consensus review as submitted through the deviation factors submission algorithm (DFSA)

85. The method of claim 84, further comprising:
xxxv) submitting deviation factors for diagnostic consensus review algorithm(DCRA) by subspecialty experts; and
xxxvi) conducting consensus evaluation by panel of sub specialty experts for diagnostic options.

86. The method of claim 85, further comprising:
xxxvii conducting consensus vote:
(a) if majority of voting sub-specialists vote in favor of deviation factors then algorithm is modified and studies and/or treatments are approved;
(b) if majority vote is against deviation factors, then algorithm is unchanged and studies and/or treatment is denied;
(c) if vote is neutral (approximately half for and half against) then algorithm is unchanged but payment is approved due to lack of sub-specialty consensus (gray area of medical thought).

87. The method of claim 84, further comprising:
xxxiii) providing through the DBME results of consensus vote:
(a) if consensus vote is against deviation factors then recommendations by DRA are confirmed;
(b) if consensus vote accepts deviation factors then DRA is modified via Diagnostic Consensus Improvement Algorithm (DCIA) to include accepted deviation factors and recommendations are altered and approved for payment.

88. The method of claim 85, further comprising:
ixl) providing through Patient Information Module results of consensus review in layman's terms prepared through layman's terms conversion algorithm (LTCA).

89. The method of claim 85, further comprising:
xl) providing through the Patient Information Module physician deviation factors in layman's terms.

90. The method of claim 81, further comprising:
xli) providing notification from the physician to the DBME of non-acceptance of therapeutic recommendations and forwarding to DBME deviation factors entered by the physician; and
xlii) submitting deviation factors for therapeutic consensus review through the therapeutic consensus review algorithm (TCRA) by subspecialty experts.

91. The method of claim 90, further comprising:
xliii) conducting consensus evaluation by a panel of subspecialty experts.

92. The method of claim 91, further comprising:
xliv) providing to the physician results of consensus vote:
(a) if consensus vote is against deviation factors then recommendations by TRA are confirmed; and
(b) if consensus vote accepts deviation factors then TRA is modified to include deviation factors and recommendations are altered and approved for payment.

93. The method of claim 92, further comprising:
xlv) providing through Patient Information Module results of consensus review in layman's terms.

94. The method of claim 92, further comprising:
xlvi) implementing modification of TRA via TCIA based on consensus vote.

95. The method of claim 94, further comprising:
xlvii) presenting to the patient information on relevant products and services relating to the patient's specific clinical situation selected through TAA including information compiled through DBME/search engine and advertisers (pharmaceutical companies, equipment manufacturers, service providers).

96. The method of claim 94, further comprising:
xlviii) providing to the physician continuous medical educational (CME) credit through CMEM and/or medical facilities and/or sponsoring advertisers and/or health insurance company to encourage physician participation.

97. The method of claim 94, further comprising:
il) providing to the physician reduced cost Malpractice coverage through the MMRMM for following recommendations of the DBME.

98. The method of claim 1, further comprising:
l) processing payment to sub-specialty members of specific clinical algorithm board identified through the MM.

99. The method of claim 98, further comprising:
li) charging health insurance carrier for processing payment authorization for approval/denial of hi tech Diagnostic test through the MM.

100. The method of claim 1, further comprising:
lii) crediting through PCPA patient's account for reduced deductible and/or co-pay based on patient acceptance of DBME diagnostic and/or therapeutic recommendations and also providing through the PCPA cost comparison information enabling patient to choose among qualified healthcare providers the lowest cost options.

101. The method of claim 1, further comprising:
liii) crediting through the MM the physician's account for submission of accepted deviation factors that resulted in modification of the specific diagnostic recommendation algorithm (DRA).

102. The method of claim 1, further comprising:
liv) processing through the MM payment to Sub-specialists participating in diagnostic consensus vote.

103. The method of claim 1, further comprising:
lv) crediting through the MM the physician's account for submission of accepted deviation factors that resulted in modification of the specific therapeutic recommendation algorithm (TRA).

104. The method of claim 1, further comprising:
lvi) processing through the MM payment to Sub-specialists participating in the diagnostic and therapeutic consensus review votes (DCRA, TCRA).

105. The method of claim 100, further comprising:
lvii) charging through the MM advertisers for ads placed on the Patient Information Module (PIM).

106. The method of claim 105, wherein the ads comprise information on relevant products and services as relating to patient's specific clinical situation

107. The method of claim 100, further comprising:
lviii) charging through the MM advertisers for receiving relevant information from advertisers (pharmaceutical companies, equipment manufacturers, service providers) and loading the information on the patient information module.

108. The method of claim 107, wherein the DBME charges through the MM advertisers based on information accessed by the patient through the PIM.

109. The method of claim 100, further comprising:
lix) charging through the MM advertisers for ads selected through PTAA and placed on Physician Action Module (PAM).

110. The method of claim 109, wherein the ads comprise information on relevant products and services relating to the physician's specific clinical case.

111. The method of claim 100, further comprising:
lx) charging through the MM advertisers for receiving relevant information from advertisers (pharmaceutical companies, equipment manufacturers, service providers) and loading the information on the Physician Action Module (PAM).

112. The method of claim 111, wherein the DBME charges through the MM advertisers based on information accessed by the physician through the PAM.

113. The method of claim 100, further comprising:
lxi) charging through the MM the malpractice insurance carrier for processing medical malpractice risk management credits for the physician.

114. The method of claim 100, further comprising:
lxii) charging through the MM the physician for billing the health insurance carrier for services rendered by physician in connection with ordering High Tech Diagnostic tests and/or providing treatment.

115. The method of claim 113, wherein the services rendered by the physician comprise one or more of patient examination, CIS preparation and submission, ordering and evaluating test results and/or providing treatment.

116. The method of claim 100, further comprising:
lxiii) charging through the MM the medical facility for billing health insurance carrier for services rendered by the facility in connection with performing hi tech diagnostic tests and/or providing treatments

117. The method of claim 100, further comprising:
charging through the MM the reading radiologist for billing Health Insurance Carrier for services rendered by the reading radiologist in connection with reading the results of the Hi-Tech Diagnostic test.

118. The method of claim 100, further comprising:
lxiv) charging through the MM the health insurance carrier for primary or overview radiological readings by qualified sub-specialists.

119. The method of claim 1, further comprising:
lxv) processing through the MM payment to sub-specialty members of specific clinical algorithm boards identified through TM.

120. The method of claim 100, further comprising:
lxvi) charging through the MM the health insurance carrier for processing payment authorization for approval/denial of treatment recommendation through TRA.

121. The method of claim 100, further comprising:
lxvii) charging through the MM the physician for billing the health insurance carrier for services rendered by the physician in connection with providing treatment.

122. The method of claim 100, further comprising:
lxviii) charging through the MM the medical facility for billing the health insurance carrier for services rendered by the facility in connection with performing the recommended treatments.

123. The method of claim 1, further comprising:
lxix) removing patient personal identifying information from clinical information by a de-coupler program and encryption identity assigned within the privacy compliance module (PCM).

124. The method of claim 1, further comprising:
lxx) Removing physician personal identifying information from clinical information by a de-coupler program and encryption identity assigned within the privacy compliance module (PCM).

125. The method of claim 1, further comprising:
lxxi) Providing patient ID/PASSWORD PROTECTION so that only patients can access their own information on the Patient Information Module (PIM) via a re-coupler program provided in the Privacy Compliance Module (PCM)

126. The method of claim 1, further comprising:
lxxii) providing physician ID/PASSWORD PROTECTION so that only physicians can access their own patients information on the Physician Action Module (PAM) via a re-coupler program in the Privacy Compliance Module (PCM)

127. The method of claim 1, further comprising:
lxxiii) providing health insurance carrier ID/PASSWORD PROTECTION so that only carriers can access their own patients subscriber information on the Insurance Authorization Module (IAM) via a re-coupler program in the Privacy Compliance Module (PCM).

128. The method of claim 1, further comprising:
lxxiv) providing health insurance carrier ID/PASSWORD PROTECTION so that only carrier can access their own patients subscriber health care provider information on the Insurance Authorization Module (IAM) via a re-coupler program in the Privacy Compliance Module (PCM)

129. The method of claim 1, further comprising:
lxxv) providing medical facility ID/PASSWORD PROTECTION so that only medical facility can access their own patients information on the Medical Facility Action Module (MFAM) via a re-coupler program in the Privacy Compliance Module (PCM)

130. A computerized method of providing information to any one of a plurality of patients for use in a medical diagnostic or treatment advice system on a computer network, the method comprising;
accessing a portion of the patient medical history during an evaluation process, wherein each patient is associated with at least one file containing medical information unique to the medical condition of the patient, selectively executing at least one medical algorithm comprising accessing a database populated with data generated through a digital board of medical experts (DBME); and providing the medical advice to the selected patient; wherein the medical advice comprises an unbiased recommendation for a diagnostic test.

131. The method of claim 130, wherein a portion of the patient's history comprising high tech diagnostics tools is accessed.

132. The method of claim 131, wherein the high tech diagnostic tools comprise one or more of CT, MRI, PET/CT and SPECT.

133. The method of claim 130, comprising digital analysis of physician provided index of suspicion.

134. The method of claim 133, wherein the clinical index of suspicion is based on an evaluation comprising symptom analysis, physical exam, lab tests and other test results.

135. The method of claim 130, comprising automatic scheduling of an optimal modality test.

136. The method of claim 135, wherein unbiased modalities are represented.

137. The method of claim 136, wherein the unbiased modalities comprise one or more of CT, MRI, PET/CT and SPECT.

138. The method of claim 137, comprising consideration of test results by Digital Board of Medical Experts.

139. The method of claim 138, further comprising insurance coverage precertification based on DBME recommendation.

140. The method of claim 137, further comprising processing deviation factors submitted by physician prior to insurance coverage precertification.

141. The method of claim 130, further comprising cost effectiveness evaluation based on clinical outcomes and costs associated with the recommendation.

142. The method of claim 130, wherein the DBME comprises members selected based on expertise in sub-specialty fields of medicine.

143. The method of claim 130, wherein the DBME comprises members who are digitally linked.

144. The method of claim 130, comprising generating a diagnosis during the selective execution of the medical algorithm.

145. The method of claim 130, comprising protecting the medical history of the patient against unauthorized access.

146. The method of claim 130, wherein the file unique to the patient stores medical information specific to the patient.

147. The method of claim 130, wherein the database is accessed through a browser.

148. The method of claim 130, wherein the computer network comprises the Internet.

149. The method of claim 130, wherein the computer network comprises an intranet.

150. The method of claim 130, comprising generating a referral to a physician.

151. The method of claim 150, wherein the medical algorithm minimizes or eliminates education and/or financial bias in generating said physician referral.

152. The method of claim 130, comprising radiological reading.

153. The method of claim 152, wherein the reading is conducted by a subspecialty radiologist who is a member of the DBME.

154. The method of claim 130, comprising an electronic notification reporting availability of the recommendation.
